SEQUENCE LISTING (40 sequences)

SEQ ID NO: 1 - DNA, 5421 bp, Homo sapiens
ccgggatccggttttttttgtttttaaaagtgtaatttcctttttatttgcatctgtttatgactgaaaaaaatgactagttattatgaagacactactgttgaagatggatattttaacatggagtttcaacaaaattacttcttgagacagagctgatgtgttttttaaataacgtgattttaagcatatatttgaacaaaactaaaacatttagtattatgaatatgaaaaaagatcagtaaatcaatgtactcttctaggctgaattaaggtagactatttaaggtttcaaaaaagtttggctggggcagaataagttttacaaaacccatgccatccaaaattaagatgacatgtagcagcaagaagtattccaatgtctcataaccagttctcgcaagcaatgtgtattccttactttaaggaagtgtcaaacaaatagaaaaatctggaagaatttactaagtgtaataaattagaggtaaatcgtaataaaagaatttatgtctcacaaaaatattcacaagtgggagttttcttttaccaacttctcagagtccttctagccccctcttcacttctgaaagatgggatttaccaaaatctggtttacatttaacttttcagggacacatgacctgaaaagaaagatgtcagataatactgacattgcctcatgcactttctttgtatcagtccttcttctgtaagtaatcagaattgggtccaaatggcatagaatcaaacattatgtatcatgccaaataccacttcctgcccaacaaaatttcatctttctccagtaatgaagaggtggacattcttgttggactgtagcatctgtgccgcccgctccacaccaaccacggcagctaacctctgggcatcatatttggagtagagaacagtgcaggtccacgtggcctcttctcctctgttggtggctctcagcatattacagatttcactgtaaaagtgtggatatgtcggcagttcatagaaaatcaggttcctgatgccttttattgctgtagtttatttccacccccttccctcctgttttctctctctccttctctctctctctctctctctcttttttttccgccctagctggggctgtgttggaggagaggaagaaagagagacagaggattgcattcatccgttacgttcttgaaatttcctaatagcaagaccagcgaagcggttgcacccttttcaatcttgcaaaggaaaaaaacaaaacaaaacaaaaaaaacccaagtccccttcccggcagtttttgccttaaagctgccctcttgaaattaattttttcccaggagagagatgtcttatcaggggaagaaaaatattccacgcatcacgagcgatcgtcttctgatcaaaggaggtaaaattgttaatgatgaccagtcgttctatgcagacatatacatggaagatgggttgatcaagcaaataggagaaaatctgattgtgccaggaggagtgaagaccatcgaggcccactcccggatggtgatccccggaggaattgacgtccacactcgtttccagatgcctgatcagggaatgacgtctgctgatgatttcttccaaggaaccaaggcggccctggctgggggaaccactatgatcattgaccacgttgttcctgagcctgggacaagcctgctcgctgcctttgaccagtggagggaatgggccgacagcaagtcctgctgtgactactctctgcatgtggacatcagcgagtggcataagggcatccaggaggagatggaagcgcttgtgaaggatcacggggtaaattccttcctcgtgtacatggctttcaaagatcgcttccagctaacggattgccagatttatgaagtactgagtgtgatccgggatattggcgccatagcccaagtccacgcagaaaatggcgacatcattgcagaggagcagcagaggatcctggatctgggcatcacgggccccgagggacatgtgctgagccgacctgaggaggtcgaggccgaagccgtgaatcgtgccatcaccatcgccaaccagaccaactgcccgctgtatatcaccaaggtgatgagcaaaagctctgctgaggtcatcgcccaggcacggaagaagggaactgtggtgtatggcgagcccatcactgccagcttgggaacggacggctcccattactggagcaagaactgggccaaggctgctgcctttgtcacctccccacccttgagccctgatccaaccactccagactttctcaactccttgctgtcctgtggagacctccaggtcacgggcagtgcccattgcacgtttaacactgcccagaaggctgtaggaaaggacaacttcaccctgattccggagggcaccaatggcactgaggagcggatgtccgtcatctgggacaaggctgtggtcactgggaagatggatgagaaccagtttgtggctgtgaccagcaccaatgcagccaaagtcttcaacctttacccccggaaaggccgcattgctgtgggatccgatgccgacctggtcatctgggaccccgacagcgttaaaaccatctctgccaagacacacaacagctctctcgagtacaacatctttgaaggcatggagtgccgcggctccccactggtggtcatcagccaggggaagattgtcctggaggacggcaccctgcatgtcaccgaaggctctggacgctacattccccggaagcccttccctgattttgtttacaagcgtatcaaggcaaggagcaggctggctgagctgagaggggttcctcgtggcctgtatgacggacctgtgtgtgaagtgtctgtgacgcccaagacagtcactccagcctcctcggccaagacgtctcctgccaagcagcaggccccacctgtccggaacctgcaccagtctggattcagtttgtctggtgctcagattgatgacaacattccccgccgcaccacccagcgtatcgtggcgccccccggtggccgtgccaacatcaccagcctgggctagagctcctgggctgtgcgtccactggggactggggatgggacacctgaggacattctgagacttctttcttccttccttttttttttttgtttttttttttaagagcctgtgatagttactgtggagcagccagttcatggggtcccccttgggcccacaccccgtctctcaccaagagttactgattttgctcatccacttccctacacatctatgggtatcacacccaagactacccaccaagctcatacagggaaccacacccaacacttagacatgcgaacaagcagcccccagcgagggtctccttcgccttcaacctcctagtgtctgttagcattccttttcatggggggagggaagataaagtgaattgcccagagctgcctttttcttttctttttaaaaattttaagaagttttccttgtggggctggggaggggccggggtcagggagagtctttttttttttttttttaaatactaaattggaacatttaattccatattaatacaaggggtttgaactggacatcctaatgatgcaattacgtcatcacccagctgattccgggtggttggcaaactcatcgtgtctgtcctgagaggctccacaatgcccacccgcatcgccattctgtagtcttcagggtcagctgttgataaaggggcaggcttgcgttattggcctagattttgctgcagattaaatcctttgaggattctcttctcttttaccatttttctgcgtgctctcactctctctttctctctctagctttttaattcatgaatattttcgtgtctgtctctctctctctctgtgtttcctccagcccttgtctcggagacggtgttttcctcccttgccccattatcttttcacctcccaggtctacatttcatggtggtcgttgggtccgcctaaaggatttgagcgtttgccattgcaagcatagtgctgtgtcatcctggtccatgtaggactggtgctaaccacctgccatcatgaggatgtgtgctagagtgtgggaccctggccaagtgcaggaatgggccatgccgtctcacccacagtatcacacgtggaaccgcagacagggcccagaagctttagaggtatgaggctgcagaaccggagagattttcctctgtgcagtgctctctggctaaagtcacggtcaaacctaaacaccgagcctcattaacccaagtgaaccaaccaaagtcaccagttcagaagtgctaagctaataggagtctgacccgagggcctgctgcttcctggttaagtatcttttgagattctagaacacatgggagctttttattttcggggaaaaaccgtatttttttcttgtccaattatttctaaagacacactacatagaaagaggccctataaactcaaaaagtcattgggaaacttaaagtctattctactttgccaagaggagaaatgtgttttatgaacgatagatcacatcagaactcctgtggggaggaaaccttataaattaaacacatggcccccttagagaccacaggcgatgtctgtctccatccttccctctccttttctgtcacctttccccctagctggctcctttggacctacccctgtccttgctgacttgtgttgcattgtattccaaacgtgtttacaggttctcttaagcaatgttgtatttgcaggcttttctgaataccaaatctgctttttgtaaagcgtaaaaacatcacaaagtaggtcattccatcaccacccttgtctctctacacattttgcctttggggatctggttggggttttgggttttttgttgttgttgtttatttgttattttaaaggtaaattgcacttttaaaaaaataattggttgacttaatatatttgctttttttctcacctgcacttagaggaaatttgaacaagttggaaaaaaacaatttttgtttcaattctaagaaacacttgcagctctagtattcacttgagtcttcctgtttttcctgtaccgggtcatggtaatttttggttgttttggttgttttcttaaaaaacaagttaaaacctgacgatttctgcagtgacttgatgctctaaaacagtgtaggatttaagaatagatggtttttaatcctggaaattgtgattgtgacccatgagtggaggaactttcagttctaaagctgataaagtgtgtagccagaagagtactttttttttgtaaccactgtcttgatggcaaaataattatggtaaaaaacaagtctcgtgtttattattccttaagaactctgtgttatattaccatggaacgcctaataaagcaaaatgtggttgtttcaaaaaaaaaaaaaa

SEQ ID NO: 2 - DNA, 887 bp, Homo sapiens
  misc_feature (139)..(139): n=A or C or G or T?U or unknown or other
aagttaaaattgttaatgaccaaacattctaaaagaaatgcaaaaaaaagtttattttcaagccttcgaactatttaaggaaagcaaaatcatttcctaaatgcatatcatttgtgagaatttctcattaatatcctgnaatcattcattttagctaaggcttcatgttgactcgatatgtcatctaggaaagtactatttcatggtccaaacctgttgccatagttggtaaggctttcctttaagtgtgaaatatttagatgaaattttctcttttaaagttctttatagggttagggtgtgggaaaatgctatattaataaatctgtagtgttttgtgtttatatgttcagaaccagagtagactggattgaaagatggactgggtctaatttatcatgactgatagatctggtnaagttgtgtagtaaagcattaggagggtcattcttgtcacaaaagtgccactaaaacagcctcaggaggataaatgncttgcttttctaaatccncagggtnaatctgggncccaancatatagacaggcttctggaaagtttgcaactggaagcaggaaacccaccntataggttaaaatcccnggccntncttgggaaaccaggtttaaaaaggccnggaanaaaaccatgccaccagggggaatccngggggtttgaggtnccctggaannncnnanaaatggngccnccngggaagggccataaagnnttttnancccantccggccttaccanaangnaaacccaatnccntttaaaaaancccgggtaaaanatnnngnaaagntggggggaaaacccaaantgggagggnnccannaaaannggtnccccaaaaacccagggggnccaaaaaaagnaanaaaa

SEQ ID NO: 3 - DNA, 1348 bp, Homo sapiens
ggcacgaggcaaagtctcactgtgtcatgcaggctggagtgcagtggcatgatctcactgcaacctccatctcctgtctcagcctcctagataactgggattacaggtgcccaccaccatgcccggctaatttttgtatttttggtagagacagagtttcaccaggttggtcaggctggtctcaaactcttgacttcaggtaatccacccaccttggcctcccaaagtgctgggattacaggcatgagccaccatcttcagccagatgatttttttattgagagagtgaaatgctattttgttccccaaatggcgctagtgaatcactaggagggtcccactgataggccatgtttagcactggttgccagggattctctttttgagagagggaaagcaaaatgaatggaagtacccagctggaggtttcagggcttctggaggatgctctcgcatagctcgaggtcctctgcccacctcttctctccaaggaaaatgaggactgccccttccccctgcaggattggcccccagcctgcgcatgcaccctcctcttgcccaagtggggagcacagaggcggagaggaatcccttaccacacccacggcccagcttgctcacgagtgtcacctctgtgacggtcaccactgctcccttggagggccacttgagttactgttgcttcctcgcctgctggcttgatgagcaccgatggtgggatctgaccccgaggggcagagctgtcggtgactgaggactggactgtggtgaccatgccgatttgctcagggagaacgttgcaatgcacccagcagctcctggctctgcaggcggcacagcctggggccctgtgatcctctggtttcttccattggggcggagtcgggggtggagggagctggccacaacccactgctctgatgggtggtttgtccaaggatgctgaatgtaatgcctggtcaatgtggaagcccatgaggttgcccagggaagcctccaaaagctgggatgcttgagggtatccaagttgaaaaagacaaaatctgaccatcagccagtgacagtcctggcaaatgaaggtggggcggggcagtgaggggtgggagaaggtgaatgattcattattccaccccgaggtttgctggggtgaggggaagaatcgatgctgctttgggaactgaaggtttttctgttgggaaggccctcttggttttggagagaaagacaagttatgagtagctgctaccctggaacggtgggcagagagcctactaggaaatgtgcagaataaactattttttgaaggaaaaaaaaaaaaaaaaaaaaaaaaaaaaaaaaaaaaaaaaaa

SEQ ID NO: 4 - DNA, 1989 bp, Homo sapiens
gggggtttgaagactgacagccagcctggctcattctcattattggctagttagctttctttatcaacctgctcactcgcaaatgtgtgccctcagccagagagtaagaaagcccaaatctgttacagcttctaaaaaaatagatttctaatttgtcctactcatgttaggagcattatctttgaaggtaaaacatagtgtatcattgtgtaaactcccaggcttgttgtagcagaagagatcatttctggaggcttcagcaatggaatttagcattataagagagattggacaaaccagtccaaagtggtccgagttcttaaatccaggtagggaactcactcttctttcttctctggacctaattgggcattgggctttagtgagaccacagaccaggcccgtctctcctgtaggcttttaattcaatggcaactctatttcaaagaataaaagcctttggagagttgcggcagttctgggggcgggctcaggagagtccatagatcagccgtaactggaacgtagaatctacgtctgcctctgaatggacttcccacctcctctctcttgctctgatgcttgcctctgggcctctccatgcccaaggtggtctttcatccttgacaggctggtaatgtgctggccacctccagctcctgcatcgagtctgtaaaccagagctggttctcatggccttcgtcacgataccaggatacggaggggagcccagggccatccatacccaccccagggtaacggggctggcctggcattagtcattatttagtttccaggccaaccatccagatagagattccctctttcctttgagcagtgctctcaagagctccgtgcctgtccacaatgacctagagtgcatcctgctcattgtcagtgtagcccctcgcccctatattcatccaggatacttggaagtgctaaaataggaagggattcggctttcaactttgctaccatcttccctgaagcaggaaaatgaacatggacttaaatgttctttgaaaaaaccaaagttttaagatttgctgtgtgatgaagtgacagggagggccggagtcagcaggtgccagactttctgttctgtctgccatgggtttgtccagctcaggtagctctaggagcaccatcctgccctagcagagcccaggccttgccctcatgaagcatcattgaaatagcaggagcatgttgatttcttggttaggttgcattataataacaagagtcagaacattaattcgaaacaacttgcagtatgcatttcttcacaccagtacattcttaagtgtacttgtttataaggaataacataaactaatctgtacctttatatatatgtgtgtgtacatatatacatatataaactgtatagtgtacatggtaatgatttattgctatgccccagatccttaatgtagttctcatcctccgcatgccctcagccacgagcgggtgactgactgttccctgatgatttggcccacctcctgtgtttggacctctagggaggagggttttggtcatactctccttatcctcgtgcacagaaatgctcagggtccccatgtgcctgttgttcagccctctctcttgttccctttctgagcatgtggtccttccccaggctgtgggacagctgccttcccacgaaagtgtaaagcagtattaagatcattactgcatgtgccctaaaaacccaagttttctattcccttaggacagaaaattgcatgtgaggtgggataatcgagtttcagtgacccacgtcagttacacattaaagccagaccccatgatgaaattccacaaaatggaaataaaactcaaatttctttagcattgtgtaaataaatctgaatgtgtttaactttgtactggtaattttctgtatatttggaatatttgggttaaaaataaaacagactggactttgttacctgacctacaaaaaaaaaaaaaaaaaaa

SEQ ID NO: 5 - DNA, 879 bp, Homo sapiens
ccaaaagttaactggctctccttcctcacacagttcatcataacccaaccccccacccccgggtcatgaaaatcacagaacttataaacacattgaaccctagatctcaggcttcctgacctaccgccagtggccccttgctggccaccctatagggtcctccttccctggcagccccccatgtgggagaatacctgattctcccaatctgcagtgggagagctttgctgaattccatcccaaagtcaaacatgggcaagaggtgaggatttcacttttaccctcaagtccgatttgtctgtgattttaaactaactgtgtatgtattgatgtttggaagattgtttgaattttaaagtgataatagtacttaatgttatccagtattgttcattaaatggtgttatcctaaagctgcacttgggatttttacctaacgctttactgattctctcaagcacatggcaaagtttgatttgcactccgttcatttctgacacgttttgctgcctcctacctttctaagcgtcatgcaaattcgagaatggagaaggacgctgccggtccctgagcggtgtggagagggcggaaggtggactccagcgcagcttgaggggctgaggacggaggctgcagcatctgtgtcgttctactgagcacgcttctctgcctcgctcctgactcagcactttgttcactggctcagcagttatgtttacacatcatttttatgttcctgctttgtaattcatgtttgagatgggtggccactgtacagatatttattacgctttccagactttctgaatagatttttttgaataaacatggttttatgaagtgtaaaaaaaaaaaaaaaaaaaaaaaaaaaaaaaaaaa

SEQ ID NO: 6 - DNA, 8500 bp, Homo sapiens
cgtcatggccgtcagcaccgcgttcccgtcctcttccgcttggccccagaaagtttcggttctgcccggcggtggacccacgagcgcgtgccaccatggagtctgaccactgctgagcagacagccaccgagggccgaaattctgagccttcctctggacccaggcaggagacatacagacaagaaaggcaaactcaccatggcctccaccaatgcagagagccagctccagagaatcatccgagacttgcaagatgctgtgacagaactaagcaaagaatttcaggaagcaggggaacccatcacggatgacagcaccagcttgcataaattttcttataaacttgagtatctcctgcaatttgatcagaaagagaaggccaccctcctgggcaacaagaaggactactgggattacttctgtgcctgcctggccaaggtgaaaggagccaatgatgggatccgatttgtcaagtctatctcagagctccgaacatccttggggaaaggaagagcatttattcgctactccttggtgcaccagaggttggcagacaccttacagcagtgcttcatgaacaccaaagtgaccagtgactggtactatgcaagaagcccctttctgcagccaaagctgagctcggacattgtgggccaactctatgagctgactgaggttcagtttgacctggcgtcgaggggctttgacttggatgctgcctggccaacatttgccaggaggacgctgaccactggctcttctgcttacctgtggaaaccccctagccgcagctccagcatgagcagcttggtgagcagctacctgcagactcaagagatggtgtccaactttgacctgaacagccccctaaacaacgaggcattggagggctttgatgagatgcgactagagctggaccagttggaggtgcgggagaagcagctacaggagcgcatgcagcagctggacagagagaaccaggagctgagggcagctgtcagccagcaaggggagcaactgcagacagagagggagagggggcgcactgcagcggaggacaacgttcgcctcacttgcttggtagctgagctccagaagcagtgggaggtcacccaggccacccagaacactgtgaaggagctgcagacatgcctgcaggccctggagctaggagcagcagagaaggaggaggactaccacacagccctgcggcggctggagtccatgctgcagcccttggcacaggagcttgaggccacacgggactcactggacaagaaaaaccagcatttagccagcttcccaggctggctagccatggctcagcagaaggcagattcggcatcagacacaaagggccggcaagaacctattcccagtgatgcggcccaggagatgcaggagctaggggagaagcttcaagccctagaaagggagagaaccaaggtcgaggaggtcaacagacagcagagtgcccaactggaacagctggtcaaggagcttcagctgaaagaggatgcccgggccagcctggagcgcctggtgaaggagatggccccactccaggaggagttgtctgggaagggacaggaggcagaccagctctggcgacggctgcaggagttgctggcccacacgagctcctgggaggaggagctagcagagttgaggcgggagaaaaaacagcaacaggaggagaaggagctgctggagcaggaggtcaggtctctgacccggcagctgcagttcctggagacccagctggcacaggtgagccaacatgtgagtgacctggaggagcagaagaagcagctcattcaggacaaagaccacctcagccagcaggtgggtatgctcgagcggcttgctgggccgcctggcccagaactgccagtggcaggtgagaagaatgaggccctggtccctgtgaactccagtctgcaagaggcctgggggaagccagaggaggagcagaggggcctgcaggaggcacagttagacgataccaaggtgcaagagggcagccaggaggaagagctccggcaggccaacagggagctggagaaggagctacagaatgtggtcgggcgtaaccagctcctggagggcaagctgcaagccctgcaggccgattaccaggctttgcagcagcgggaatcagccatccagggctccttggcctccctggaggccgagcaggccagcatccggcacttgggtgaccagatggaggcgagcttgctggctgtaaggaaggccaaggaggccatgaaagcccagatggcagagaaggaggccattctacagagcaaggagggcgagtgtcagcagctgcgggaggaggtggagcagtgccagcaactggcagaagcccggcacagagagcttagggctctcgagagccagtgccagcagcagacccagctgattgaggtcctcacagcagagaaaggccaacagggagttggcccacccactgacaatgaagcccgtgagctggctgcccagctagccctgtctcaggcgcagctggaagtccatcagggggaggtccaacggctgcaggctcaggtggtggacctccaggccaagatgcgggcagccctggatgaccaggacaaggtgcagagccagctaagcatggctgaggccgtcctgagggagcacaaaacccttgtgcagcagctgaaggagcagaatgaagcccttaacagagcccatgtccaggagctgctgcaatgctcggagcgtgaaggggcactgcaggaggagagggccgatgaggcccagcagagggaggaggagctgcgggccctgcaggaggagctgtcccaggccaaatgcagctccgaggaagcacagctggagcacgctgagctgcaagagcagctgcaccgggccaacacagacacagctgagctgggcatccaggtttgcgcactgaccgtggaaaaggagcgagtggaggaggcactggcctgtgctgtccaggagctccaggacgccaaagaggcagcctcaagggagcgagagggcctggagcgccaagtagctgggctgcagcaagagaaggagagcttgcaggagaagctgaaggcggccaaggcagcagccggctcactgcctggcctgcaggcccagctcgcccaggcagagcagcgggcccagagcctccaagaggctgcacaccaggagctcaacaccctcaagttccagctgagtgctgaaatcatggactaccagagcagacttaagaatgctggtgaagagtgcaagagcctcaggggccagcttgaggagcaaggccggcagctgcaggctgctgaggaagctgtggagaagctgaaggccacccaagcagacatgggagagaagctgagctgcactagcaaccatcttgcagagtgccaggcggccatgctgaggaaggacaaggagggggctgccctgcgtgaagacctagaaaggacccagaaggaactcgaaaaagccacaacaaaaatccaagagtattacaacaaactctgccaggaggtgacaaatcgtgagaggaatgaccagaagatgcttgctgacctggatgacctcaacagaaccaagaagtatctcgaggagcggctgatagagctgctcagggacaaggatgctctctggcagaagtcagatgccctggaattccagcagaagctcagtgctgaggagagatggctcggagacacagaggcaaaccactgcctcgactgtaagcgggagttcagctggatggtgcggcggcaccactgcaggatatgtggccgcatcttctgttactactgctgcaacaactacgtcctgagcaagcacggtggcaaaaaggagcgctgctgccgagcctgtttccagaagctcagtgaaggccctggctcccctgatagcagtggctcaggcactagccagggagagcccagccctgcactgtcaccagcctcacctgggccccaggccacaggaggccaaggagcaaatacagactacaggccaccggacgacgctgtgtttgatatcatcacagatgaggaattgtgccagatacaggagtccggctcctctttgcctgaaacacccactgaaactgattctcttgacccaaatgcggctgaacaggatactacatcaacctcgctaacgcctgaggacactgaagacatgcccgtggggcaggattcggaaatctgcctgctgaagtctggagaactgatgatcaaagtacccctcacagtggatgagatcgccagcttcggggagggtagcagggagctgtttgtgaggtccagcacctacagcctgatccccatcactgtgcccgaggcagccctcaccatcagctgggtcttctcctctgaccccaagagcatctccttcagtgtggtcttccaggaggccgaggacacaccgctggatcagtgtaaggtcctcattcccacgacccgatgcaactcccacaaggagaacatccagggccagctcaaggttcgcacacccggcatctacatgctcatcttcgacaataccttctcaaggtttgtctctaaaaaggtattttatcacttgacggttgatcggcctgtgatctacgatggaagtgatttcctgtagcttcagcacctcagtaacttcacttcatccacaggaaacactgctcttcctcacctgtcacataaagcatttttttaaaaagtcagctgctccaaaatcatcaactcagcccctgggctgcccctcagaggcggtgtctggggaggactttgtgctcagcactctgcaccggccactcttagcccccgaggcgttgaagggctcaggcaatgtttccattaagtagagactcagctgttgtcacacccaaagggatgctctgccaaaggtttaaacacccaggagaccatcagcctctcctgggagcacagttgactacaggcctcttgtggagagtttcacgggcaggggtgattccaacttctgcctgtggagagattttctgccctgccccaccagggccctgcatgttggagactgagctgggtgcactggccataccctgtgaatcctcgggctgtgacgccctcaggtactcctgggaaaaggaggtacacagccatcatgcgagtcggtgccaggggaccccccggagatcctgaccagctcctccagtcatgctcttgtccctcactgccccagtaagctggaggctgctccagaactcagcagtgttggaggggcctctaagctgcactctctttctggcccttttgtctggctgattctgtcctcaaataaagcccttcactcagccagacctctccacagctcaaagcattgccctaagaatcagaagtaaagataatccaagagcaaaacccactgtacttggggcctgcaatggctgtgtgtacactacatctaatgcccaaatgccagccagtgtggatgttgtgaccacagagcaggattgtgcattggctttagagctactcctcagctgatggcccacttttgtttatataaataagagcttctgccccacctgcagacatgtttactaatgatcatagccaggattagaaccactttcaaacattggggccttcttaacaaaagtctttgataacttaagaaccaaagtaacagagtaaacagaggcatgatggatccctgggccccactcccctcctgacaggttccccaacagcccatttgcccacttcccactgctcagcccacaccagacctccaggagacatccccccttgaggcagagagatcctgttccctattcccagacaagaattatttaatcttccctgttctctgtggtccttttcttccccaacaacagatagctcaccttggacagctcttcgtcccttgttcatggaaccagctgcctgcagtcaggccccaggttcttccatgggtgaacagagcatctgacaaaaggtcccagtttggccaggggtgagggagagagcaccagacaggctatccgagaatctgagagctgggcccggcaattcctccagctacccttgtgacctaagtccagtcacacatttcccaaagtttctctttgtcataaccctggtctggctggttttgagggcttgagaatgggtcagggactccaggccaagtccaacagagaccccaaacccaccacacaccagcagccacaacctcaccaccaacaaagaggacttttgtggggccacaagtaagaggtcatttctggaatggactcagacctttaaacaggagagttgagcacttccagtcagtttttaagcaaggcatggggaacagggaatagaacctttcaaagaggttgcccagagaaaagctgggcctcttgcattcggcttccttggagcagcctcttctggcagaaagccatcaggtgctcaatcatcttctcctggccaaggctctgaccatgcttagtactggaatagaggtggccaggcccccagcgactcttcttggcctgatgtttgtcctcacaggcatgccacgtggcctgagatgattcagaacaaatcatgctaactttgaatccatccagccacttgcaaatgataatcagaagtcagcttgttcactgttagaaagaaactaacaaaagagaacccagagcaatctagaatctttgagtgcttggctttccaaggatactgcggagactctggccaagctgatgaccttctgaagtgtcactggcaccatatgcaacaagaaccaccattcactgagtagctaatgggtttggggcctgggacattccatctgaggtccttcctgaacatgtcactccacagcagaggaccggttgcagcttacccagaaccactcctccaggagagctggatgttttgcgtgcaacaccttgagcactgactgctattgttcaaaaaaagcctttgctgcattcggaggactgccccgtgccctgaggtgacttcctaactatgtggtttcattagcgaatttattttttgggctgggtggacatttgtattttgttaggttgctgtttaagctcaagtttgctgtgctctctgcagctacaaaacatcttggcatatttaagagtggcttttataaatagctttattctgatattaatcagattcccaactttactgagaattaaggactggggtactttaaagaaatgcaaatagcaattgaagaaccactgctgcaggtggtagccctggctagactgaattacactagaaatcagccagaaggaagcgtccttgggatcccagatcactcttttttttttttttttttaaaaggggcagccccttgatggctcatctctctgaataacagttacgtcttcatatcgataccagatgccttcttcatcatgccactgaagccactcaccaccttcaagaacatgccaacctctgtcagattcacttacccacaaacaaggaggcacgtttggcacaaagtgttgtcctccaggtccaagtggactctacagagtgcttgacctcaacacactggattccaggtggactggaccaagagcaggcaaagacacgggaactgaaaaactccacagggtttggagaatagaaatgaaaagccacgtcatataactcaagaataaatggtgttttggaaattttaaaattatcatcgaaggtggtgaaactatttcaggcccaaatgaaaggaaatcgccagttggggatgaaatcacagagcctgtgttttatgatatggttggatgtccactgatgaaattttaaaggagtttcatttttaaaagtgcgcatgattctacatatgagaattctttaggccaagaaactgtccttggctcagaggtgttgggaattaaagcagagagaagccattcgtgatgcttagaaccaaggatggtcatgtacacaaagaccatcgagacggccattcttgtttacaaaacacttaccaagaaagcactttgtaggggaactttagtaagttcttctcatttcattatgtttcttccaaggaaacaggagagactgaattaataattctctctttcctcttaagcacttttaaaataataaagtacatcttgaaatttggggaggcatctctgatttaaaaaaagaaaaaggctgcttgatgtatgttatgcagagacactctgcctctggtggctgcagagcaatacccaagcctcatttggaaggctcaacatttggaattgcactttaattgattaatcctcaattcatgtggccttacgggatggtgggtctgggaccccaattcattcttatctgccaaagaattatctagaagcacatcaaataccagcaccccacctgcacaatgggggtggaaaacttttgtatccctaagcatattattttatagtgtctgccatgccatgtggaaatactttatttttaacctcaggatttaaataaagtaaacactatgacattt

SEQ ID NO: 7 - DNA, 6289 bp, Homo sapiens
gttggatttctctaatggaaaatgttattcagaaggatgaagataatattaaaaattccataggttacaaggcaattcatgaataccttcagaaatataagggttttaagatagacattaactgtaaacagctgacagtggattttgtgaaccagtccgtgctacaaatcagcagtcaggatgtggaaagtaagcgtagtgataagactgattttgctgagcaacttggagcaatgaataaaagttggcaaattctgcaaggtctagtaactgagaagatccagctgttggaaggcttattggaatcttggtcagaatatgaaaataatgtacaatgtctgaaaacatggtttgaaacccaggaaaagagactaaaacaacagcatcgaattggagatcaggcttctgttcaaaatgcactgaaagactgtcaggatctggaagatttgattaaagcaaaagaaaaagaagtagagaaaattgagcagaatggacttgctttgattcagaacaagaaagaagacgtctctagcattgtcatgagcacactgcgagagctcggccaaacctgggcaaatttagatcacatggttggacaattaaagatactgctgaaatcagtgcttgaccaatggagtagtcacaaagtggcctttgacaagataaacagttacctcatggaggccagatactctctttcccgattccgtctgctgactggctccttagaagctgtgcaagttcaggtggacaatcttcagaatctccaagatgatctggaaaaacaggaaaggagcttacagaaatttggctctatcaccaaccaattattaaaagagtgtcacccacccgtgacagaaactcttaccaatacactgaaagaagtcaacatgagatggaataacttgctggaagagattgctgagcagctacagtccagcaaggccctacttcagctttggcaaagatacaaggactactccaaacagtgtgcttcgacagttcagcagcaggaggatcgaaccaatgagctgttgaaggcagccacaaacaaggacattgccgatgatgaggttgccacatggattcaagattgcaacgacctcctcaaaggactgggcacagttaaagattccctctttgttctccatgagctgggagagcaactgaagcaacaagtggatgcttccgcagcatcagctattcaatcggatcaactctctttgagtcaacacttgtgtgccctggagcaagctctctgcaaacagcagacttcattacaggctggagttcttgattatgaaacctttgccaagagtttagaagctttggaggcctggatagtggaagctgaagaaatactacaagggcaggaccctagccactcatctgacctctccacaatccaggaaaggatggaagaacttaagggacagatgttaaaattcagcagcatggctccagatttagaccgtctaaatgagcttggatataggttacccttgaatgataaggaaatcaaaagaatgcagaatctgaaccgccattggtctctgatctcctctcagactacagaaagattcagcaagttgcagtcatttttgctacaacatcagactttcttggaaaaatgtgaaacatggatggaattcctagttcagacagaacaaaagttagcagtagagatttcaggaaattatcagcaccttttggaacagcagagagcacacgagttgtttcaagccgagatgttcagtcgtcagcagattttgcactcaatcattattgatgggcaacgtcttctagaacaaggtcaagttgatgacagggatgaattcaacctgaaattgacactcctcagtaatcaatggcagggagtgattcgcagggcccagcagaggcgggggatcattgacagccagattcgccagtggcagcgctatagggagatggcagaaaagcttcgtaaatggttggttgaagtgtcctacctccccatgagtggtctcggaagtgttcctataccactgcaacaagcaaggaccctctttgatgaagtgcagttcaaagaaaaagtgtttctgcggcaacaaggcagctacatcctgactgtggaggctggcaagcaactccttctctcggcggacagtggcgctgaggccgccttgcaggccgaactcgctgaaatccaagagaaatggaaatcagccagcatgcggctggaagaacagaagaaaaaactagccttcttgttgaaagactgggaaaaatgtgagaaaggaatagcagattccctggagaaactacgaactttcaaaaagaagctttcgcagtctctcccggatcaccatgaagagctccatgcagaacaaatgcgttgcaaggaattagaaaatgcagttgggagctggacagatgacttgacccagttgagcctgctgaaggacaccctctctgcctatatcagtgctgatgatatctccattcttaatgaacgcgtagagcttctgcaaaggcagtgggaagaactatgccaccagctctccttaaggcggcagcaaataggtgaaagattgaatgaatgggcagtcttcagtgaaaagaacaaggaactctgtgagtggttgactcaaatggaaagcaaagtttctcagaatggagacattctcattgaagaaatgatagagaagctcaagaaggattatcaagaggaaattgctattgctcaagagaacaaaatacagctccaacaaatgggagaacgacttgctaaagccagccatgaaagcaaagcatctgagattgaatacaagctgggaaaggtcaacgaccggtggcagcatctcctggacctcattgcagccagggtgaagaagctgaaggagaccctggtagccgtgcagcagcttgataagaacatgagcagcctgaggacctggctcgctcacatcgagtcagagctggccaagccaatagtctacgattcctgtaactcggaagaaatacagagaaagcttaatgagcagcaggagcttcagagagacatagagaagcacagtacaggtgttgcatctgtcctcaacctgtgtgaagtcctgctgcacgactgtgacgcctgtgccactgatgccgagtgtgactctatacagcaggctacgagaaacctggaccggcggtggagaaacatttgtgctatgtccatggaaaggaggctgaaaatcgaagagacgtggcgattgtggcagaaatttctggatgactattcacgttttgaagattggctgaagtcttcagaaaggacagctgcttttcccagctcttctggggtgatctatacagttgccaaggaagaactaaagaaatttgaggctttccagcgacaggtccacgagtgcctgacgcagctggaactgatcaacaagcagtaccgccgcctggccagggagaaccgcactgattcagcatgtagcctcaaacagatggttcacgaaggcaaccagagatgggacaacctgcaaaagcgtgtcacctccatcttgcgcagactcaagcattttattggccagcgtgaggagtttgagactgcgcgggacagcattctggtctggctcacagagatggatctgcagctcactaatattgaacatttttctgagtgtgatgttcaagctaaaataaagcaactcaaggccttccagcaggaaatttcactgaaccacaataagattgagcagataattgcccaaggagaacagctgatagaaaagagtgagcccttggatgcagcgatcatcgaggaggaactagatgagctccgacggtactgccaggaggtcttcgggcgtgtggaaagataccataagaaactgatccgcctgcctctcccagacgatgagcacgacctctcagacagggagctggagctggaagactctgcagctctgtcggacctgcactggcacgaccgctctgcagacagcctgctttctccacagccttcctccaatctctccctctcgctcgctcagcccctccggagcgagcggtcaggacgagacaccccagctagtgtggactccatccccctggagtgggatcacgactatgacctcagtcgggacctggagtctgcaatgtccagagctctgccctctgaggatgaagaaggtcaggatgacaaagatttctacctccggggagctgttgccttatcaggggaccacagtgccctagagtcacagatccgacaactgggcaaagccctggatgatagccgctttcagatacagcaaaccgaaaatatcattcgcagcaaaactcccacggggccggagctagacaccagctacaaaggctacatgaaactgctgggcgaatgcagtagcagtatagactccgtgaagagactggagcacaaactgaaggaggaagaggagagccttcctggctttgttaacctgcatagtaccgaaacccaaacggctggtgtgattgaccgatgggagcttctccaggcccaggcattgagcaaggagttgaggatgaagcagaacctccagaagtggcagcagtttaactcagacttgaacagcatctgggcctggctgggggacacggaggaggagttggaacagctccagcgtctggaactcagcactgacatccagaccatcgagctccagatcaaaaagctcaaggagctccagaaagctgtggaccaccgcaaagccatcatcctctccatcaatctctgcagccctgagttcacccaggctgacagcaaggagagccgggacctgcaggatcgcttgtcgcagatgaatgggcgctgggaccgagtgtgctctctgctggaggagtggcggggcctgctgcaggatgccctgatgcagtgccagggtttccatgaaatgagccatggtttgcttcttatgctggagaacattgacagaaggaaaaatgaaattgtccctattgattctaaccttgatgcagagatacttcaggaccatcacaaacagcttatgcaaataaagcatgagctgttggaatcccaactcagagtagcctctttgcaagacatgtcttgccaactactggtgaatgctgaaggaacagactgtttagaagccaaagaaaaagtccatgttattggaaatcggctcaaacttctcttgaaggaggtcagtcgtcatatcaaggaactggagaagttattagacgtgtcaagtagtcagcaggatttgtcttcctggtcttctgctgatgaactggacacctcagggtctgtgagtcccacatcaggaaggagcaccccaaacagacagaaaacgccacgaggcaagtgtagtctctcacagcctggaccctctgtcagcagtccacatagcaggtccacaaaaggtggctccgattcctccctttctgagccagggccaggtcggtccggccgcggcttcctgttcagagtcctccgagcagctcttccccttcagcttctcctgctcctcctcatcgggcttgcctgccttgtaccaatgtcagaggaagactacagctgtgccctctccaacaactttgcccggtcattccaccccatgctcagatacacgaatggccctcctccactctgaactaagcagatgccatctgcagaagtgctggtagcataaggaggatcgggtcataagcaatcccaaactaccaacaagaggaccttgatcttggcgaaagccctcggtgtggcagctttagccctcctccagatcacatgtgtgcaaattatggcttcagaggtggaagataaacagtgacgggggaacaaacagacaacaagaaggtttggaagaaatctggtttgagactctgaaccttagcactaaggagattgagtaaggacctccaaagttccccggactcatgaattctgggcccttggcccattctgtgcacagccaaggacttcagtagaccatctgggcagctttcccatggtgctgctccaaccatcagataaatgaccctcccaagcaccatgtcagtgtcgtacaatctaccaaccaaccagtgctgaagagattttagaaccttgtaacatacaatttttaagagcttatatggcagcttcctttttaccttgttttcctttggggcatgatgttttaacctttgctttagaagcacaagctgtaaatctaaaaggcacttttttttagaggtataaagaaaaactagatgtaataaataagatcatggaaggctttatgtgaaaaaagttgaatgttatagt

SEQ ID NO: 8 - DNA, 1041 bp, Homo sapiens
ggcacgagggaagttggacgcatgcgccgtttctctgcatggtgtgcgttctcgttctagctgcggccgcaggagctgtggcggttttcctaatcctgcgaatatgggtagtgcttcgttccatggacgttacgccccgggagtctctcagtatcttggtagtggctgggtccggtgggcataccactgagatcctgaggctgcttgggagcttgtccaatgcctactcacctagacattatgtcattgctgacactgatgaaatgagtgccaataaaataaattcttttgaactagatcgagctgatagagaccctagtaacatgtataccaaatactacattcaccgaattccaagaagccgggaggttcagcagtcctggccctccaccgttttcaccaccttgcactccatgtggctctcctttcccctaattcacagggtgaagccagatttggtgttgtgtaacggaccaggaacatgtgttcctatctgtgtatctgcccttctccttgggatactaggaataaagaaagtgatcattgtctacgttgaaagcatctgccgtgtagaaacgttatccatgtccggaaagattctgtttcatctctcagattacttcattgttcagtggccggctctgaaagaaaagtatcccaaatcggtgtaccttgggcgaattgtttgacaaatggcaactgacttctttagaattttgcagttaacagtagtatgtactcaaattggggggaaaaaaaccctacatgtttcttgtaaaggcgtctgacagtcctgagaattattgatggtaaggaataaaaaatgtacagatgactcagtgaagaaactgaggcttctcttatgaaacaaacattgataaacgtaactactaaatgtttatgcctctgtaaaccaaatttcttttctagataaaaatatgtattactacctgcaaattttcttctggctgttttagtagtattttttttacagaactaaatatagagtttgtatgattagtaaaaaaaaaaaaaaaaaaa

SEQ ID NO: 9 - DNA, 721 bp, Homo sapiens
  misc_feature (386)..(386): n=A or C or G or T or U or unknown or other
ttttgtttggcccaaagtaaacatgtttattctcagttctgccttaggggtctctagttttgcaagcatgagtaaatggaatcaacaataatcctctccttaaatgtctggcattaaaatttgtcacttaagaagtttcctgttttgcctaaagagagtgtgatttgagggtgacctgaaacaaggcttgaggcttgtggacacatagggttaatcgccttatttcctgccaaatcgcagagcagtgaaaggccaaaggaagctataaatagcagcccggcagatctgtccttccaagagggaaagaacttagcaacaaagagagacacgaggggtgaagtgggcaaagaatcattagcccagtttctgcccatgccagggcatgntgacccttgggaatgctgcgaggcccagcagaggaagaagaggatcaaagctttcataacctccaactcagtgcatcccaaacccagacgggcctggaccgacctgtgcatttactcctgaatgccctcagtcagcagacacgggagccatcaggtggggaaacgtgtcctcagagtgctccttttttttgaagtggacacagctccagccaggaatggcagagaggaaaggatcctgcaatngagtggcttctgtcttcagggttcacagacagtcttcgatgacccatgagntgtttggcgctcagcttcatcggcgggcctctctggcttgg

SEQ ID NO: 10 - DNA, 478 bp, Homo sapiens
ttttttttttttttgtttttttttggggatttcccaatgattttattgaggtaacttttcccaattttatacatatatgcatttatatatacttaggaaagctaaacaatgttctaaggcacttggaattgtgcacagcaaagtatcctctaatattatacaactaaatagagcagaattttgctttttaaataacacaaataccagtacggaattaaaaaagggaatacatagtctttctttcaggttacaatagtggaatacaagtacatatgtgtgtatacttgtagatatttatacccacatactataatacagtacagataagaacaacaaaagagaaactgtcatgttaatcagtgtacagttccagttatttacactcacagatattacacctgtgtaatacgtagaactagatcactcactggaaatcagaaagcattcagtcagtctgataatgatcacagtttaccat

SEQ ID NO: 11 - DNA, 439 bp, Homo sapiens
  misc_feature (11)..(12): n=A or C or G or T or U or unknown or other
taaaaaaaacnnctgaatttttttccacccacaaacacatggaaagtgcagaaaccagttaatctatgtgatgtatttgcatacgtttacaaacaagacaaattaaaacagaaacatgttcagaatttaacctgattaaatattaagttcagtcctgagcttttgatatttaagacaatatagataaagcaatagcaaaaaattttaatttatttgatttgcatgctacagagatttaggctaaactttgttcatttgggctaggcaatattctttttgtacctggtaacactttagggttctggatattacaaaattggtaattaattatactgggattaatttccaaacttggggggacttaaatatttaccattcctttttttacccctggtggcggggattaaattccttacccntttttaaaaatgggggattt

SEQ ID NO: 12 - DNA, 595 bp, Homo sapiens
  misc_feature (14)..(14): n=A or C or G or T or U or unknown or other
gccagagctggcanggggaagttgctaaaggatgtcttccggcctggggannttttcttcaacactggggacctgctggtntgcgatgaccaaggttttctccgcttccatgatcgtactggagacaccttcaggtggaaagggggagaatgtggccacaaccgaggtggcagaggtcttcgaggccctagattttcttcaggaggtgaacgtctatggagtcactgtgccagggcatgaaggcagggcatggaatggcagccctagttctgcgtcccccccacgctttggaccttatgcagctctacacccaccgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcanctgagagaggactctgtggggttgggggccg

SEQ ID NO: 13 - DNA, 525 bp, Homo sapiens
  misc_feature (353)..(353): n=A or C or G or T or U or unknown or other
cagcttaaaacatgtctctgcattttattttaagaaaacacaaacctggtcacaaaacatcttcagagaacaggataagtgaagaaacaaacaaaatgcatgggaatagcaaatttggggccacggtcatgttcaaggggcggagctgacatgacatcattttgttcccggaaaagcaaggttaactcaagctgtgaagcccagatggccaatagatttaccggccttctaaggaagagcagaatgctctcaagagctgaattatgatgacttgtaggtattgattagatgagaacaccaaccccatattcagcagagagttagggaatgagaagtagaggggagaatgtggngaaatcgnctntatgtanttttcaaagtcacttccgaaaaagaagatgaggtaatagttgaatatgccagcaacggacatgaggaacaggaagagtatgatgcgtttcccaaatatgtagccaggggtgctctgcgttctctctcctaggtaaccgacaaagtacttttgcc

SEQ ID NO: 14 - DNA, 680 bp, Homo sapiens
tttttttttttttttttttagttgtgttacgagcttttatttagaaagcacatttaatacaagtatagtttcgcagatacaagttttcactttgtatgctacaaaagtctttgaatattattctctttacaaaatggaaccttacaaaaatactgacaatttaatgtttttatacagttttctctagttgcagttatttcattataaaacaatgtctaccacagaactatgatattttagttgatatttaaaaaaattaactcaatgcttttttaagcagctaatgtaaataacacaggtcgagacacagtttataatcatagtggatatagctaaattgtttcagaaataatatcttacatagttaacttttaatgttttatacattatttatataatatttatatataaaaatcatagcttgctataagttgaaatgaaaggacggattctgtagtaaggatgtgcatgtggttgatccatatggttacatttaaccctttgaaaggtctgcatccaagatctaaacgcatttcttccttcctcctttcctcaaaggttcagtagaaggggtccctgtctatcacctagagtgggaccttgcatggaaggacacttacggatagaggatgaggaaaactctctaccgagatttaacccatatgttctgcccagcagaa

SEQ ID NO: 15 - DNA, 5460 bp, Homo sapiens
cgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcaaggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggcccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcagctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacagggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaata

SEQ ID NO: 16 - DNA, 455 bp, Homo sapiens
tttattatcaacagacaaaaaaagtttattgaatacaaaactcaaaggcatcaacagtcctgggcccaagagatccatggcaggaagtcaagagttctgcttcagggtcggtctgggcagccctggaagaagtcattgcacatgacagtgatgagtgccaggaaaacagcatactcctggaagtccacctgctggtcactgttctcatccaggctgcccatcagcttcttcagcccctcctcatccactttctcccccacaaagctgggcagctccttgtgcagaagttccttcatttcccccttactcagcttgaacttgtcgccctcttggcaggagtacttgtggaaggtagtgaccagcacagccagcgcctgctccagagaactgcacatcatggatctgtggctgtgtacttgttttctctcagcctcacccccacatggtgagctcac

SEQ ID NO: 17 - DNA, 2420 bp, Homo sapiens
ggatccaggccctgccaggaaaaatataagggccctgcgtgagaacagagggggtcatccactgcatgagagtggggatgtcacagagtccagcccaccctcctggtagcactgagaagccagggctgtgcttgcggtctgcaccctgagggcccgtggattcctcttcctggagctccaggaaccaggcagtgaggccttggtctgagacagtatcctcaggtcacagagcagaggatgcacagggtgtgccagcagtgaatgtttgccctgaatgcacaccaagggccccacctgccacaggacacataggactccacagagtctggcctcacctccctactgtcagtcctgtagaatcgacctctgctggccggctgtaccctgagtaccctctcacttcctccttcaggttttcaggggacaggccaacccagaggacaggattccctggaggccacagaggagcaccaaggagaagatctgtaagtaggcctttgttagagtctccaaggttcagttctcagctgaggcctctcacacactccctctctccccaggcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttcactggctcttttcttctccatgcactgagcatctgctttttggaaggccctgggttagtagtggagatgctaaggtaagccagactcatacccacccatagggtcgtagagtctaggagctgcagtcacgtaatcgaggtggcaagatgtcctctaaagatgtagggaaaagtgagagaggggtgagggtgtggggctccgggtgagagtggtggagtgtcaatgccctgagctggggcattttgggctttgggaaactgcagttccttctgggggagctgattgtaatgatcttgggtggatcc

SEQ ID NO: 18 - DNA, 5826 bp, Homo sapiens
  misc_feature (4852)..(4852): n=A or C or G or T or U or unknown or other
ggggggggggggggtgggagcgtggttgagcggctggcgcggttgtcctggagcaggggcgcaggaattctgatgtgaaactaacagtctgtgagccctggaacctccactcagagaagatgaaggatatcgacataggaaaagagtatatcatccccagtcctgggtatagaagtgtgagggagagaaccagcacttctgggacgcacagagaccgtgaagattccaagttcaggagaactcgaccgttggaatgccaagatgccttggaaacagcagcccgagccgagggcctctctcttgatgcctccatgcattctcagctcagaatcctggatgaggagcatcccaagggaaagtaccatcatggcttgagtgctctgaagcccatccggactacttccaaacaccagcacccagtggacaatgctgggcttttttcctgtatgactttttcgtggctttcttctctggcccgtgtggcccacaagaagggggagctctcaatggaagacgtgtggtctctgtccaagcacgagtcttctgacgtgaactgcagaagactagagagactgtggcaagaagagctgaatgaagttgggccagacgctgcttccctgcgaagggttgtgtggatcttctgccgcaccaggctcatcctgtccatcgtgtgcctgatgatcacgcagctggctggcttcagtggaccagccttcatggtgaaacacctcttggagtatacccaggcaacagagtctaacctgcagtacagcttgttgttagtgctgggcctcctcctgacggaaatcgtgcggtcttggtcgcttgcactgacttgggcattgaattaccgaaccggtgtccgcttgcggggggccatcctaaccatggcatttaagaagatccttaagttaaagaacattaaagagaaatccctgggtgagctcatcaacatttgctccaacgatgggcagagaatgtttgaggcagcagccgttggcagcctgctggctggaggacccgttgttgccatcttaggcatgatttataatgtaattattctgggaccaacaggcttcctgggatcagctgtttttatcctcttttacccagcaatgatgtttgcatcacggctcacagcatatttcaggagaaaatgcgtggccgccacggatgaacgtgtccagaagatgaatgaagttcttacttacattaaatttatcaaaatgtatgcctgggtcaaagcattttctcagagtgttcagaaaatccgcgaggaggagcgtcggatattggaaaaagccgggtacttccagagcatcactgtgggtgtggctcccattgtggtggtgattgccagcgtggtgaccttctctgttcatatgaccctgggcttcgatctgacagcagcacaggctttcacagtggtgacagtcttcaattccatgacttttgctttgaaagtaacaccgttttcagtaaagtccctctcagaagcctcagtggctgttgacagatttaagagtttgtttctaatggaagaggttcacatgataaagaacaaaccagccagtcctcacatcaagatagagatgaaaaatgccaccttggcatgggactcctcccactccagtatccagaactcgcccaagctgacccccaaaatgaaaaaagacaagagggcttccaggggcaagaaagagaaggtgaggcagctgcagcgcactgagcatcaggcggtgctggcagagcagaaaggccacctcctcctggacagtgacgagcggcccagtcccgaagaggaagaaggcaagcacatccacctgggccacctgcgcttacagaggacactgcacagcatcgatctggagatccaagagggtaaactggttggaatctgcggcagtgtgggaagtggaaaaacctctctcatttcagccattttaggccagatgacgcttctagagggcagcattgcaatcagtggaaccttcgcttatgtggcccagcaggcctggatcctcaatgctactctgagagacaacatcctgtttgggaaggaatatgatgaagaaagatacaactctgtgctgaacagctgctgcctgaggcctgacctggccattcttcccagcagcgacctgacggagattggagagcgaggagccaacctgagcggtgggcagcgccagaggatcagccttgcccgggccttgtatagtgacaggagcatctacatcctggacgaccccctcagtgccttagatgcccatgtgggcaaccacatcttcaatagtgctatccggaaacatctcaagtccaagacagttctgtttgttacccaccagttacagtacctggttgactgtgatgaagtgatcttcatgaaagagggctgtattacggaaagaggcacccatgaggaactgatgaatttaaatggtgactatgctaccatttttaataacctgttgctgggagagacaccgccagttgagatcaattcaaaaaaggaaaccagtggttcacagaagaagtcacaagacaagggtcctaaaacaggatcagtaaagaaggaaaaagcagtaaagccagaggaagggcagcttgtgcagctggaagagaaagggcagggttcagtgccctggtcagtatatggtgtctacatccaggctgctgggggccccttggcattcctggttattatggcccttttcatgctgaatgtaggcagcaccgccttcagcacctggtggttgagttactggatcaagcaaggaagcgggaacaccactgtgactcgagggaacgagacctcggtgagtgacagcatgaaggacaatcctcatatgcagtactatgccagcatctacgccctctccatggcagtcatgctgatcctgaaagccattcgaggagttgtctttgtcaagggcacgctgcgagcttcctcccggctgcatgacgagcttttccgaaggatccttcgaagccctatgaagttttttgacacgacccccacagggaggattctcaacaggttttccaaagacatggatgaagttgacgtgcggctgccgttccaggccgagatgttcatccagaacgttatcctggtgttcttctgtgtgggaatgatcgcaggagtcttcccgtggttccttgtggcagtggggccccttgtcatcctcttttcagtcctgcacattgtctccagggtcctgattcgggagctgaagcgtctggacaatatcacgcagtcacctttcctctcccacatcacgtccagcatacagggccttgccaccatccacgcctacaataaagggcaggagtttctgcacagataccaggagctgctggatgacaaccaagctcctttttttttgtttacgtgtgcgatgcggtggctggctgtgcggctggacctcatcagcatcgccctcatcaccaccacggggctgatgatcgttcttatgcacgggcagattcccccagcctatgcgggtctcgccatctcttatgctgtccagttaacggggctgttccagtttacggtcagactggcatctgagacagaagctcgattcacctcggtggagaggatcaatcactacattaagactctgtccttggaagcacctgccagaattaagaacaaggctccctcccctgactggccccaggagggagaggtgacctttgagaacgcagagatgaggtaccgagaaaacctccctcttgtcctaaagaaagtatccttcacgatcaaacctaaagagaagattggcattgtggggcggacaggatcagggaagtcctcgctggggatggccctcttccgtctggtggagttatctggaggctgcatcaagattgatggagtgagaatcagtgatattggccttgccgacctccgaagcaaactctctatcattcctcaagagccggtgctgttcagtggcactgtcagatcaaatttggaccccttcaaccagtacactgaagaccagatttgggatgccctggagaggacacacatgaaagaatgtattgctcagctacctctgaaacttgaatctgaagtgatggagaatggggataacttctcagtgggggaacggcagctcttgtgcatagctagagccctgctccgccactgtaagattctgattttagatgaagccacagctgccatggacacagagacagacttattgattcaagagaccatccgagaagcatttgcagactgtaccatgctgaccattgcccatcgcctgcacacggttctaggctccgataggattatggtgctggcccagggacaggtggtggagtttgacaccccatcggtccttctgtccaacgacagttcccgattctatgccatgtttgctgctgcagagaacaaggtcgctgtcaagggctgactcctccctgttgacgaagtctcttttctttagagcattgccattccctgcctggggcgggcccctcatcgcgtcctcctaccgaaaccttgcctttctcgattttatctttcgcacagcagttccggattggcttgtgtgtttcacttttagggagagtcatattttgattattgtatttattccatattcatgtaaacaaaatttagtttttgttcttaattgcactctaaaaggttcagggaaccgttattataattgtatcagaggcctataatgaagctttatacgtgtagctatatctatatataattctgtacatagcctatatttacagtgaaaatgtaagctgtttattttatattaaaataagcactgtgctaataacagtgcatattcctttctatcatttttgtacngtttgctgtacnanaaatctggtnttgctmttmnactgttaggaagaattancatttcattcttctctagctggtggtttcacggtggccaggttttctgggtgtccaaaggaagacgtgttggcaatagttngggccctccgacaagccccctctgccgcctccccacagccgctccanggggtggctggagaacgggtgggcggctggagaccatgccagagcgccgtgagttctcagggctcctgccttctgtcctggtgtcacttactgtttctgttcagggagagcagcggggcgaagcccaggccccttttcactccctccatcaagaatggggatcacagagacattcctccgagccggggagtttctttcctgccttcttctttttgctgttgtttctaaacaagaatcagtctatccacagagagtcccactgcctcaggttcctatggctggccactgcacagagctctccagctccaagacctgttggttccaagccctggagccaactgctgctttttgaggtggcactttttcatttgcctattcccacacctccacagttcagtggcagggctcaggatttcgtgggtctgttttcctttctcaccgcagtcgtcgcacagtctctctctctctctcccctcaaagtctgcaactttaagcagctcttgctaatcagtgtctcacactggcgtagaagtttttgtactgtaaagagacctacctcaggttgctggttgctgtgtggtttggtgtgttcccgcaaaccccctttgtgctgtggggctggtagctcaggtgggcgtggtcactgctgtcatcagttgaatggtcagcgttgcatgtcgtgaccaactagacattctgtcgccttagcatgtttgctgaacaccttgtggaagcaaaaatctgaaaatgtgaataaaattattttggattttgtaaaaaaaaaaaaaaaaaaa

SEQ ID NO: 19 - DNA, 33023 bp, Homo sapiens
cttgcctcagcctccccatagctgggagcacaggtgcgtgtcaccgccccagctaatttttaaattttttgtagagacaaggtttcgctatgttgcccaggctggtctcgaacccctgggatcaagtgatctgtgtcaggcctctgagcccaagctaagccatcatatcccctgtgacctgcacatatacatccagatggcctgaagcaactgaagatccacaaaagaagtgaaaatagccttaactgatgacattccaccattgtgatttgtttctgccccaccctaactgatgtactttgtaatctcccccacccttaagaaagttctttgtaatctccctcacccttgagaaggttctttgtaatttgtaattctccccacccttgagaatgtactttgtgagatccacctcctgcccacaaaacattgctcctaactccagcgcctatcccaaaacctataagaactaatgataatcccatcaccctttgctgactctcttttcggactcagcctgcctgcacccaggtaaaataaacagccttgttgctcacacagagcctgtttggtagtctcttcacatggacgtgtgagacaatctgcccacctggtcctcccaaagtgctgggattacaggtgtgagtcaccaggcccagccgagaaagagttgaatacacgtagaggagactggagttttattattactcaaatcagctgccctgaaaatttgaaggctgggatttatttatttattttttatttttttgagatggagtctcgctctgtcgcccaggctggagtgcagtggcatgagctaggctcactgcaagctccgcctcccaggttcaagcgattctcctgcctcagcctcctgagtagctgggattacaggcccgtgccaccacacctggttaattttttgtatttttagtagagacggggcttcaccatgttagcctggatggtcttgatctcctgacctcgtgatccacccatgttggcctcccaaagtgttgagattacaggcatgagccaccgtgcccagccctgaaggctaggttttttttttgagacagagtctcactctgttgcccaggctggagtgcagtggcacaatcttggctcactgcaacctccacctcccgggttcaagcgatttccggctaacttttgtatttttagtagagacaggggtttcaccatgttggccaggctgctcttgaactcctgacctcaagtgacccacccgcctgggcctcccaaagtgctaggattacaggtgtgagccaccgcacccagcccgaaagctaggattttttaaagatagtttggcggacagggggctagggaatgggtgctgctgactggttgggtgggggatgattcttgtgtgctgagctgagtctgcttttaggtagggccacaggaccttgagtcataggtctatgtggtccgggtggagccatctggtagtgagaaatgcaaaaacctgcaaagacgtctcaaaaagccaaccttaggttctacaatagtgacattatctacaggagtaattggagaagttacaaatctcttgagctctgaacaatggctggtcatcatgaatgcttccatgttagcagaattcaggcccctctcatcctcctcacctgatggcctttcattacttttacaaaggcggtttcatcttgggaaggtctgttatcatttaaactataaacgaaatttctcccaaagttagcttggcccatgcccaggaaagaccaaaaacagtttggagggtaaatgcagacagggttggttagatcagctctctcactggcagaattttgttactgttacagtttttgcaaggcagctttagggtgatgggtctgcacggaatatatgcatgtaacagaaccgcgcttgtaccctctcaatctataaaacaaataaaaccagcctaataaaagtttacataaaatgtaaaaaacaaagcaaagcctcctttctgcgggtctgtgtaaacgagcacagctggtgggaagggcgcgggtggggggttctgctgccccccatccctgccctgctgcaggccctcgcccccagccccattctttctgttctccgcttggctgcagccgcacgtcggccccctccccaggagctggaagtacaaagcccttccaggtggactctggctccccctttgttcccagcttattctaattccaaagctcattgtgcccggctcgccttcagaagaggaggcgcccccatcctgtctccagctgcccatcctcccaggataaccagtcaccccagggcccggtggcccctcacccagcccctccccggtccgcagctgccctaggcttgagtgggcgctggctccaattctcaggcctcccccaacaaacaggagcattccggctagcccccctcccctgccctccccccagctccccttctcctcccctctccctcctcctcagctcctactccaaccccccagccccagctggggcctgaaaggctgcccactccctgggacacggtaaggggagggtgcagctctccccccgcccctccccggtcgcctctgccccagagaacagtttgcttctcacccagaagccaccataggagctctgggctgggcacaggtcgcagggcacccccaccccctcctgcacatgctcggaaccccccttcagtgagtagaacacaagggcctggcaagacaggcggaggcttggaaagggctggcgggggacagctacccggccctcagctgggggcctggagagcccaccctgcccctccccagcagctgctgcccccgggccgagcctgacgcgccttgacaaagcccgagaacgctttgaagccttcggacgtgggagaggacccagccagggatggaaatcgctttgcctttgttcccccaactgctcagcagctcgtgggagcagcctcagaagaccctgtcactggccgcccgggtcagagcctgtgacagagaccagagctccggccggagctccccgccggaaactccagccttaccagcctgaacttcatgcactgctcaaagaggcccagctcgccctggctgggcaggggcggctcccagggtggccgacgtggggagggttcctgaacctccctcaggagcctcctgggacagagtcctggaggtcagcagagaaagggaagcccaggctgtgggccccgcgtggagggaaaactcagggggaacgccccgaggctgggagggcacagccccatcacactccatctcgtagtcttggagaaaaatttagttctacctccagggcacagaaaagcccaaagaagggatgagataaaggaggggctggtcagatttgtgcttcaggagggtccctctggctgctcagagaatgcagtttggggtctggcccaggccgtgaggaggagttcacagtcactatggggtccagaccagctgggggcgggaggcagggcctggcccaggggctggtagtgggtgcaccccaggctgggctggcaaagggcggggaagaaggtgggtgtgggaaggggtggctgcctggagaagagcccagttccaggaggtcgtgggagtcaggtatgggggcccactggacctcccctcagatgtgggggctcccactccgagggtccatggagcagtggcagccattctggacagccccccacccttcacttctgtctccagtaccctccagcctggccacctcgccctctgcctcggcctcctctgatctcacaaaggcagcagcagtcaggggtggcgactccctcatgatcctgtccctggcctcggaacccaccggaggagtaaccgcagcagtggtcacttccccaaagtgccatcagctctcctacatccttctcgagcctctgctttctctcccttccacccccacccctttgacaagcaacatctgaaagtctcccctgcccgcggctcccagagctgtctggccgtggcctgcactctccctcaaagcggccctcccccaggtcaccgtcctcccttcgatgacatcacgcgcccaccccgcactcctgctgggctgaggccctccgagcctacttcaccgggtcctcttctttctctcaaaggcaatggggtttgcctgtggtccagctgggttggccctcttccctctgtcctgggtccttgagtggccccgcttctttaggccatctatgagtcacttctctaatgcccctgtctccagaccagcttcagtcaaaggctgggccagagaagaccctagtgagaaacttctgatgagcagtgtgaccttgccacctcaggggtacccacccaccacccctggtctaagcacaggtgacaccgcctgtctcccccaaccacacacaccccttgaggctcctcctccaagcctgggtggggacactgtccctccctcacccagcaagctcaatctggcttgggccggaactgcttttcttcctaaagctggacggatggccgcgggcttagcttaacgggatgagccatctggggactgcagtgtccacgatcagatcagggagcttgaagctgaggggggcacactttacctcccaggccaggacaatgaccacttccttccccaccccacccccaggctactcttagccctagaaaattctaaacaagctgctcagctggcggcggagaggcagcccaacaagctggctcttgctagggaggcctggggggtcctggggagaggaacacggggtgggtggggggcgggcagccaggacctcaggcctgaggcctttggggaagggtctgtgcacctgccaggcaccagggggcagccttgccttgttcccgctccagtcccctcaagtccgaagcccctacccactctcacgccaggcaggggtgggggccgccggggtcatttacccgggccccttctctgccttgatgacaaagtcgagccttgctcatcagccaggcaggctcccctctgcccactgtggagacacagaggcctgtcacctgaagagctggtcccggcctccagcttccagggtagccgggaagctgtagcccccagtgggcagcggtggagagagctcaaggaaggagggagcaccgggaggagacggctgcagcctgccaggagcggggagaaagggagagaaggggaggcggagggctgagggggcccgggggacgtcttcccagggctgggaggggccggccgggaagcctgggctgcactaggagccggcgaccctggggcgaggggcggcccggagccctgcgggaggagctggcggccgccccaggtagcaaccatcctgcctcccgctggagcggcgtctcctccccgggaggagggcagggaggaggtgggcggagtgtgacgaggagggcgggagggagggatgcgggagggggagggggaggggggccggccggccgtgggggtggggcgatagtgacatcaccccggagtcggtttttaagcggcggccggccggggacggggaagagagggatagtcggagcgaggtggcgagtcgctgagcccgccgcggccccgagagcggctgcagccgccgccgccgggaaggagagggcgaggcgcgcccgagccgccgccgccgccgccaccgccgccgccgccaccaccgccaccggagtcgcgggccagccgggcagcctccgcgggccccggccggggcggggggcgcgggccacaggcccctgctccggccgtcgtttgcagaccgcgggcgccgatgtcgcccgcgccccgttaggatgagtctcgggtcgggcgaggagccgccgcagccgccgccgcccgagccgcgggcaggagcctcgggagccgccgccgccgccgccgccgcccggccgggccccgacgccgcccgcgcgcccccgggcccccgacacacatgagattcttcaggctcactttcaagtgcttcgtggactgcttctgactgcgccgcccgcgccccgcaccccgccgtccgcccgccgccccgtcccccggcccggccgccccccggcccccggccggcccgcgccctcggggccctccccggtgccgccggtgccccccgcctgaccgccgccccccgtgaggcgccgcgaccccggcccggccgtgcggcccgccggggccatggcgaagaagagcgccgagaacggcatctatagcgtgtccggcgacgagaagaagggccccctcatcgcgcccgggcccgacggggccccggccaagggcgacggccccgtgggcctggggacacccggcggccgcctggccgtgccgccgcgcgagacctggacgcgccagatggacttcatcatgtcgtgcgtgggcttcgccgtgggcttgggcaacgtgtggcgcttcccctacctgtgctacaagaacggcggaggtgagttcccccgcccgccgcggcctcctcccccagcaggccgccggcccccgcccgacccccggagccgccgcggaggggtgaagtccgggcaacgggtggcccccgggcacgcgggggtcggggccgcccctcgtccgccgctgccgctcggtggccgggccgggcgcctccacccccctcgcagtcatgtgcctggcatggtggggggagggggccggcgatgcccgcgaggctgccccccagactcccgggctgggaggagcgattggccgccgaggtgggaaagcaggcctgcgccttggggtctccgcgaggtaaggagccctggctgcccccacgggtcgggcacacaagcggcacattgtgtgggccccccacgtgtgcacacacacgaacacacacacacacaatgggccactctgtccctccccctgccctcccctcccctcgcggccctcccgcccctcccctctggcccgggcctggaacactgggtgcccgagccaggcttgggaagcctgcggcctggcccgcctggcgccgccactggacacactgcatgcacgtcccatgcccgcccgcccgcccgcccgcccgggcccagcttagcaacagcgatgggcacgcgtgtgtcctgtgactacaaaacagcactggggttgctggaagccgaagtgacccggtgatgggtgggaaacagaggtccagagcaaaggcctttgcccaaggtcaggagaaggatgctgggacctggagtcaggcaagttgcagccaagctcagcctctgagtagtggagcgagcccagccagggcaagggtaggaggcccagagaggagaagggggtagtggcacccagctctccctgcccttctgccacccccaccccagcctgctggcctcaggagataggcctgtgtcacgccctgcctatctcctgcagagcctgactccctggccttgctaaggccggcctggcccctcttccgcacctgtatccctctgtccttgcacatcgccatcccaccagcaggggactgtgacccacccaccctctgccttagacctcacacttgcaggcaagcgtccaagggcaggacagtcgcgctccctgcctttggatgagccccccaggcctgatcacccagccttggcacacatgcacacatgcacgtgccctcactgtgctgcctgaaacagggaattgcagcactagggacagcccgcgtgtctgagcgtgtgtgtcctccatggccatcgccccaagtgaccgtgggggtggaagccctgggggcctagggcccctctgccacccagggaatagggctccaatggctcaggggctactgtagcccctcttcaacacactcaacccaccccctcaagactccacctggggcctgagtcagtggccacccctacactgactcacccagtcggaagttgtgatggggcctttggagtctgggctggcccgctgggcctgggcagcctggctgggggccaccctgagtccacgctgtgcctccacccccaggtgtgttccttattccctacgtcctgatcgccctggttggaggaatccccattttcttcttagagatctcgctgggccagttcatgaaggccggcagcatcaatgtctggaacatctgtcccctgttcaaaggtgagcagcccttggccagcctcagggactgcccccttctcccagctggctcccacttgagaaatcttttcctgtcgtgagcaccaggcctggggccacgtgatggcgtcccagtctcgaggggggagcctggaggagatgttcaggccgcacagcgaacttggggaagcggggactagagggggcataggcagctccacaaggcaaggacaggccaggcatagccgggctggggacgggacctgcccagcagcacccttggctctctaggtaggtcctactgttactatccccaaggacgctggggcacagacaggtggagcgacgtactgaggttgcccactgcaggggcgactgtctccaacactacctcaggcgactagaaacccccccccccccaccaccaccatcaacaccagctgctgaggactggaggctactgggtggccaggcagaggcttggacctcctggaaccgccatggtggcagtgggacccacagaaggggccaggtgtatgaggctggagactccacagcacttggtcagatggggacaggaggagaggggctcgctctgccttgggtctagggggcggctggaggagaggagacaggctggggagtcagcgcagtgttggggctcacacaagggggagcccaggggagtcaggagcaccacaaacaaggctccaggaggacagatggtgggagcacggccagcctgggtggggacataaaggggtggcagggggaggtggccagggaagaatctacatggcaaggacttcccggccccaggcctgggctacgcctccatggtgatcgtcttctactgcaacacctactacatcatggtgctggcctggggcttctattacctggtcaagtcctttaccaccacgctgccctgggccacatgtggccacacctggaacactcccgactgcgtggagatcttccgccatgaagactgtgccaatgccagcctggccaacctcacctgtgaccagcttgctgaccgccggtcccctgtcatcgagttctgggagtgagtccggcacctctgggccaagcccatcccatcccccaggtctccctcatgttgcccggctccaggggagtggccctgagggggcaccagggtgttgcctggcagtccatcctggaccctgcctgcccttgcctgtcctcggagagtcctggggccagcctcgctcctgggttcggcagccgatcactgtcctggtcactcccccctgatgggggagctggggctgcatgtgaggtgggatgggagtggcctcccaatggccaggggatcgtgggctccaggcccagcccaattggacaagagggacccgctgaaccctgggctgtgggagagaagggagccacaactcctgggggtggaccctgtggctccatcctctgctggcacaggcctcatgggacctccctccctcccctaggaacaaagtcttgaggctgtctgggggactggaggtgccaggggccctcaactgggaggtgaccctttgtctgctggcctgctgggtgctggtctacttctgtgtctggaagggggtcaaatccacgggaaaggtaccactagaggcatgcagcggggagggtggctcagccctgggagccggatgtctgtgccaggcacacctgtggcaacgggaggtgaccagacagagtctagccctaaggaagggggaggtactgaaagccaagcaatgctccccaccctgcaaatccagggcccagcagcctttgctcctggggatagaggccctggcaggcactgtcccttccctgtgcccatcacccccactggtgccctcctgccagtctctgactcttgtgacagtctggtggacctggtctggccatctgttacctatcttgccttggggacccagagcagagtctggccacatcccttgggggctcctggtcaggctggggagtcacctgaacaaagaagacagtgtctagagctgtgggacatggccagctccctgggggacaaggtccccagagcagcatgtgggaagagggggcagacagtgtggcagctgcatctcgcctgcctctgcctggcccagttccactctccacctgctcaacccccacctctctccagaagaggagggggacccgacccggatccaatatcccgctccctgcctgggcctcccacacctgcactgcccacacactcatacagctctcactccccacgtgctccacgcctcctgtccccactgaggagagctcccagaggctcgcctgctccccaccgacacgcgtccctgcagacaaacgaggcgcccagggagcttccccactgcacttggccagggctgccggggcgcagccttgcccctagcttcctctggcgggagccatggctcggaggacaatggggacctctgaacatacctgcccgcaagggggaccggaggcgctgggagtgggggtgtgagggaggtggtgccacagcctccgctgagcagcctggccccccagatcgtgtacttcactgctacattcccctacgtggtcctggtcgtgctgctggtgcgtggagtgctgctgcctggcgccctggatggcatcatttactatctcaagcctgactggtcaaagctggggtcccctcaggtgaggtggaggtggagaggctgcagcagggcgctgcgggggagccctgcaggcccctcatgcctgcgctctccggcccttctctaggtgtggatagatgcggggacccagattttcttttcttacgccattggcctgggggccctcacagccctgggcagctacaaccgcttcaacaacaactgctacaagtaagcaccgccgccctgccacccgtgccctgtcctgccctgccccgccctgcccagcagcctaacccatccactctggcccctccacccctcagggacgccatcatcctggctctcatcaacagtgggaccagcttctttgctggcttcgtggtcttctccatcctgggcttcatggctgcagagcagggcgtgcacatctccaaggtggcagagtcaggtagggccctacccccagccccgcctccagagcagcgagtgctacccagatgcatgatgtacaggaacatgcaatagaaatgctgaaaagtgacgaggattcaaacggaacttgtcagattgtgggcctgtgggggcaggtcctgggatttgtcaatgttgacagagaaaggacctcccagcccctgccgcacgacccagggttgacagcgcctctgaggcaggcgtgggcatgggcgcgagtgttgcaggcagggctcagggtgcgcacagggcaggacatcggctacaaggtctagagcctgcacctttcccacagggccgggcctggccttcatcgcctacccgcgggctgtcacgctgatgccagtggccccactctgggctgccctgttcttcttcatgctgttgctgcttggtctcgacagccaggtttgcatggggctctgggacagggagccaggaggggggcggagggagggctgcaggcaaggaaaggggtggagggcggtgcggggctcggcctgagctgccctggccacagtttgtaggtgtggagggcttcatcaccggcctcctcgacctcctcccggcctcctactacttccgtttccaaagggagatctctgtggccctctgttgtgccctctgctttgtcatcgatctctccatggtgactgatgtgagtggggtggggggtctgcctgtgacctctggtggccgtctgccatcctccctgactgggctctgtcccccagggcgggatgtacgtcttccagctgtttgactactactcggccagcggcaccaccctgctctggcaggccttttgggagtgcgtggtggtggcctgggtgtacggtaggtcatggctgagggctgggctgggggatggtggcggggaaggcaggtctccagcttggccctcccgcctcacctcgccgcaggagctgaccgcttcatggacgacattgcctgtatgatcgggtaccgaccttgcccctggatgaaatggtgctggtccttcttcaccccgctggtctgcatggtaagggctgggggaggtggggcagggcggggggcgaggcagggcggggtaggggccccattaaccgcagcattctggtccgtagggcatcttcatcttcaacgttgtgtactacgagccgctggtctacaacaacacctacgtgtacccgtggtggggtgaggccatgggctgggccttcgccctgtcctccatgctgtgcgtgccgctgcacctcctgggctgcctcctcagggccaagggcaccatggctgaggtaaggctcccgcccggcccgccctcccctcccctgctgtgaacattcaacccagcctgcttcctagccagggagtggccccgactagggtggcaggcagtgggaaccggagagaggcagaggaagtcaccgtggggacgagcaggtgaccctgggggcttcagcatgtcctcctctcctgcagcgctggcagcacctgacccagcccatctggggcctccaccacttggagtaccgagctcaggacgcagatgtcaggggcctgaccaccctgaccccagtgtccgagagcagcaaggtcgtcgtggtggagagtgtcatgtgacaactcagctcacatcaccagctcacctctggtagccatagcagcccctgcttcagccccaccgcacccctccagggggcctgcctttccctgacacttttggggtctgcctgggggaggaggggagaaagcaccatgagtgctcactaaaacaactttttccatttttaataaaacgccaaaaatatcacaacccaccaaaaatagatgcctctccccctccagccctagccgagctggtcctaggccccgcctagtgccccacccccacccacagtgctgcactcctcctgcccctgccacgcccaccccctgcccacctctccaggctctgctctgcagcacacccgtgggtgacccctcaccccagaagcagcagtggcagcttgggaaatgtgaggaagggaaggagggagagacgggagggaggagagagaggagaagggaggcaggggaggggcagcagaaccaaggcaaatatttcagctgggctatacccctctccccatccctgttatagaagcttagagagccagccagcaatggaaccttctggttcctgcgccaatcgccaccagtatcaattgtgtgagcttgggtgcgagtgcacgcgtgcgtgagtacggagagtatatatagatctctatctcttagcaaaggtgaatgccagatgtaaatggcgcctctgggcaaaggaggcttgtattttgcacattttataaaaacttgagagaatgagatttctgcttgtatatttctaaaaagaggaaggagcccaaaccatcctctccttaccactcccatccctgtgagccctaccttacccctctgcccctagccaaggagtgtgaatttatagatctaactttcataggcaaaacaaaagcttcgagctgttgcgtgtgtgagtctgttgtgtggatgtgcgtgtgtggtccccagccccagactggattggaaaagtgcatggtgggggcctcggggctgtccccacgctgtccctttgccacaagtctgtggggcaagaggctgcaatattccgtcctgggtgtctgggctgctaacctggcctgctcaggcttcccaccctgtgcggggcacacccccaggaagggaccctggacacggctcccacgtccaggcttaaggtggatgcacttcccgcacctccagtcttctgtgtagcagctttaacccacgtttgtctgtcacgtccagtcccgagacggctgagtgaccccaagaaaggcttccccgacacccagacagaggctgcagggctggggctgggtgagggtggcgggcctgcggggacattctactgtgctaaaaagccactgcagacatagcaataaaaacatgtcattttccaaagcaggctcctgcttccgcctctgctgctctaaggaaggggtcggggtacaggaggcagggggaacctcctccagctggagctgctgccgtgagcaaggctctgctctggaggcctctgcggccggcacccttctggggactgggaagggggcagggaaggcagcagcccaggggaaggccttgtccccctggagccgaggcagttggggagagcaggacgagagtgagctggagagcagccacacccgcggggaagggtgggcgtaaagccatgggtgctgaaattttcaaaatgttaccccaagaatttgtcactgaacaggtgccttgtgtcacttgggccaggctggtagcagcagaggggataactctgcatcagggatcaattttgaaggtggagccaataggggttgtgcatgaccaggatgcagggctcaaagaggagttaaggacaacagatttggcctgagcaagaggaaagatggagctgccaggtcctgcaatggggaggcaaggagagaatggtctggagtcagccttgggtgtgtcatgcaggaagtgtcatccaagtggagatgtctagttggcaggtggacacaggagttccagaaagtactggagatggaactgtgcaagttcttaccacatagagatgacactgaaagccctgagcctgagtgagctcacagggacgccgcaagccccggaacacaatgagaggggcagagcgaagacgtggcagtgataggggaggacgcctgagagttcctggtggggtcctgcaacctgagccagtgaggacccctcacaggtcagggaggagcagtggctggctccatctgtccagtgctgctgctggtgaaggacagtgacctgcaaatgctcactgagtctggcaagggtcacgggggcctggcgagggtggcttgcatgagcgggtgcgtgtgaaaggctgggtggtgtgcgactgagaaaaggagtggcggcagcgcagtgtcatctgcagacgaagggagagacaacaacgtagttcacccagacaaggaaatatgagccagcctggaaagggaaggcattccaacacacgacacaacatggctgaccctggagggcatttctgtgaaatgagccatcataaagggatacttgctatagggttctgctcctgtgagagagacagggccttacatgagaggagggagatccacagagacagagggcaagggtgggtgccaggggctggggacagggtggggagtgttgagtggggacagagtgtcagtttgagaaaataaattctagaggtggatggaagtggtggctgcgcaacactgtgactgcacttaatgccactgaattgcacatttaacgatggtgaaaatggctcattacatatacactgatgacactatatatatgtatgatatatatgcgttttaccatgagaagaggtggagaggaattggagacactgagtacagacaggtccttcaacgggcgggaccccgtgcacaagatgagcatgtggcaccccaccctcaaagggctgggcaccatggcagggcacagcaggcaatgcagtgggcggctcaggcaagcacagagagcatcagagattggagcctgtgaagggggagcaggtgacccctcagagcaaagtgacagcttgggctgctccctttgcgtcctgcccaggactgctatcgtgctatgggagaacccccagaggccctgctcctcagcaggcagcaccccctatggaggggctttacccctaaacttctggagccaggggagggacctggcttggaatacggccaaccaagagcctgggtgagaaatacacggaccagacagggagcagagaaaggagtggcagtgcagtcccaccctagctcagccaggggctctggagcctgtcctgcagtccctggcccccatctcttcagcaaccgctgtttccagttttctttttctccctgagaagcctgtcctctcaccatgcctgcgccttcaagaaccccgcctgctggcagctcccacatctccggcctggccctccttagctgcaaaggtgcttcccaacatcagcaagacctctccccagggtgccccaggccctcacacagcccctgtccccaaccgactccaactgtcctgcagcccacagtcaccctcaggacccctgagctcaggccaactgctttatacactgtcagccaagtctctgcctggatgacaatcaccctctgctaattgttctccgcacctccaggccaaatgccctccaagccacctcatgcaccacgatgacactaaacacacagaaaaaagacattgaaaaaaggaaacttcacagaggcctgtcacttaaagagggtcctgaaatagagacaccatttcctcaggacttagctcctgcatcaggggttaggacacagagatcaacaagcagcaggctttgccctcaagcagctcgcagtctagtggaagatgggtaagaaaacagatcaggacgcccacgggtgcagatgccctggaacagaagctgatccaggaaggcgcgagctgcaggccgccctccagtctaggctgggcaagcacctcaattttcatctctaagagcctgtgcccacaccccctgccccgttgttgttccatcactccactagaaagggcgctccagaagctggcctcgtgcagctttctgtctgctgctggcctaggcagaacagcggaagaagccatcagggctggtgagggaagcacccgtttggactttagcctttcaaagctcagagaagggtgagctcagggaggtccaaggtagctgagagcacttcctggaaaagtgggatcagccttcggccttggcacagcaaccagagggtatcgcccacgtgtcccctactccctcagacaccacctctcagaccgcctggaaagggacagaactcgtcatgaggcggctgtgctctgagcacaagggaagggcgacaggatgctagagaagggaaccactggcctgggcccggacagggcaggcagaagcgagcatgcacagcaggccgtcagctaccctgccagcatcaacatccttcaggggtccccccagttccaggagacacacctctaacctgctcccctgacccttccgcccagtcctcatgcagacaccaggcatggcagaggccctgcagggtggaagcactgtgctgcgggcgggggctgccttcctcatgtgctactggagagtagcacagtgcaggggcctgggcactggtgccaggcaggaagccccggtactggcctggcttgctgtgggcctggaagacacagctctgagggagccacgggagggacaccctggagccagcacagcgctctggtggcaggcacacacccagcacgttctcagggccaagggccccagcccattcccagcccctttctgcctagctctgccctgggccagctccaggtcactgccaaggacaagtctcctctcccagctggcattagtcagaggtcatcctgcaaaccttcgggggggggggcagggagtgactagtggcgttctgccacgttctgtctgtcccaaatgtgacgaacaggaacccagagaaggcaagcgagtcctctacccggaagccccgccggtttactgagcctcccaagctgcccacacccagggaggcagacaggacacacactcggcgggtggccctgaagcgaggcctggcccagcccggggagcaggaggacagagagggcaaggccttcgagaacaggtgtgagcctggccttcagtgggggaaacaggttgaagggctgtggccgcttgggggctccaggcaggagagaaagcagagccctccccacagctgcagtcacacaccgcaccacgtacacaccatgacaacttttattgccctcaagagaaactccagtccacctgctccacccaccctcctgcgggaccaaagaaaacacccagagggcaaaacaaaaaggggctcaaaccaacaggaagtcagccccaccgcaagccggactacaactaactcgtgctctccacgctcaggcgtggaagccaaggctgtgccaggcctggccaggccaagcaggatgacagcaaacgcattctgaacgtgtagcaatcaggtcccctgtaatgtgcttggagagtgtggacaagggccgagatgacgagctatgagctgtggaagggaatgggggaagcagaagggcacaaacagaagtactggagggagaggccaggctctcaggaagcagcaggcacgtgccaggtggaagccagctgcaggcaggggaggaaggaggcccttactcttccttcttgtccatgggaccatctactgcagcctggaaagggacagaaatcccacagcagtaggttggccgggtccactcctcccctgccacctccagccccatgccccagaggtccacctcggttcccctctctcctaacaacagctattcaagtgaacaaggggccccctccccagctgcacccaaaggcctgccagggtgggagcgtcagccctggcccacgctctagggaaagccctggacctaacgccagccagggaggactgccaggacctcactgggggctgagtcctggctgcagggaacagcaaggcatccagtccccttcaagacctgatcagacccttcccaactctgcacacctttgaacaggtgccctcgaagcccatctgccaagcctgccccatacagagggcatgggtgccccctttgaggctggacccttcctccccacctgctgtggtgcccaaacttgggccaccaagcactgaggccagctgtccaaagttaggagtatttatgtggccctcactcccaacgtcaagaccgcctgggcttccagatgcggcctggtgcacccaagctagtctgaggactcagatcaggcctagggcagcaggtgatggccacaactagcgcctgctagggaaggtgcctttttgacaccttgtgccctcacttgcccagggatctttgccctacgtcactccccagcaccctaggaaagaaggccagcagtgggtcccagagtttcacctgcttctttgttcttgaccaggccccaaaccatggctgcgcctgagcacgaaggtaggaaggctcagagcctagtgagccagtgccactcctgagggccgccttggcaagtgcctacatctgctgccaggccacccccctcctgcccggtgaagggtcccactcagtagggcagaggtggccagggggagtggtggagagggcagccagcccctgggcccctggaaggttccctccgcacccgcaggggctgcctcatcctgctctgctctcctgccctgggcgcagcaatacgggagggctgacctgcagctttgcgtgctcctccagcaagcggtcgtactccttggtgaggccctcagactgcttccgcatggccagaacctggttttcagctttctctagttcttgaaatgatgtaaatgaccaagaaaacagaaacgaaaagacaggaattagggggaaaaaacccgactgctacagacaccagaaactggcccaaatctatctcaaacgaggttatacaggaggctacttctcaaaataaagcccctctgcttttgcaggcccccaaagtagagggaaagggctgacaaaaaagctcaagataaagcaaaagaaacacagaggccatcccccagtccctttaatggagaggaactctagtggctctcggcaagggtaacctccagggaggctgagagtgggagacagggagcaagatcccagcctgcaagcgagacccaatgacaaccacgccttgcacacagcagcagcaggcgaggcctgtggtattgggggaaaacgccccagacttaagtctatgcgtgggagaccaaagacaggcaggccgcttgggagccgcccactcccctcctgaacgccactcccacactcccctcattctcagcccccaggcatgctggggctaccgtgccacactctggacgggaaagccccagcatgcactgctctagtgcagggcaatcgaggcccaccaactgcagcctggttcctcctgagccccattcaaaccacttagcctcactggcctgccggctaagcatggctgcattggggttggaggcgcagggtgctattggtctgttttcagccagccctcgagcgtgcgtgcaaggcttgttactaatactttggcacaaaatgggcagcagcgggcagaggaggctcctctggacttccctgcggggaaggacacgaggtcgagcctcactttgcttagtgctggccagctcgtcctttagcttctgcaggtcagccttcaggctcctgttctcttcctccaacttcacctcagcattcccgacatccaacttgcctccgtcaacagcagctccctgggaaaagtgccaaaggccagggttactcaggagggagggagggagaggttccagccccatcctccccaccgagctgcggttcctcaagctgccctggccacacgccccttcggaaatgtcaacgcggaaccgagccaccacttgctcccagctcctaggcaaaggccagggcgtggctgcccgccgagggaaagagaagcgccagcggggccacctgctgcagctcgccgggcacgccttgcctgccctggcccctggcccctggcccctggccctgcctccttcccaagcagcagggctcagcagctccatggtgctcaccaacccctccacagatggcggtgcctcgtgctccctacatggtgccgctcactgcagttaggagcccccagtcggcctggccagctctatcccacctctgcatccacatccctccgagcttgccttgcagctcacctcctgacgggacgtctaagactggccaactaccctgcccccacctcctctccagcactgagggatgccacagaccccgagttccagagggggtgcggcaatcttgcagggaacaagggcctagctgagggccttcggatcacagcagagggcctggctcactgaggggccatttttctcagggaagggtctaactggaagcagtggatggaaacgagagcagcaacaccctcctcctcacccggaccctcacacacagacgcctccagcaggcatactctccccactgaggacttcccctctgcgcctccacccaactctggcttttcaggcacatttcccagcgtgacaggctagcagtggccactgaggccctgaagaatgtggctcccacagtgtaacaccaggacgccccatggtgggtcgggaagctgggctcaccttcttgagctggtcattctcctccatgtacttcttggccgcctcactagcactctccgcctgctttttaaaggcttcattggaggccagcagcgtggcctgctgcgaaatgagagtcaccaggcgtctaagcaggctgcaattatttacaaaaagaagggagaagtgagaaaaagagcatgaagggctggcaggagcacctcctggttgctcccactccacacctagctccagcctggacctgccctcttgccaaggcagccgagtgagaagccgccaacctggtgctggcagcgtgagggaaaaggtggggcccaggagccgtcctctgccgctgtgcccaacggccaccctcagctctcagaggggctggaagcaggagcctggggggctgggaagagcctcgctacagcatgaggtcccagaacgcggcactttccgggtcggggcctagacgtgccagacaagccacagcaccaccttcctccctgcgaggctgggcttgccttggtaaggtaacgagagaagctaatcaatccaagcacttccaacatgccaggccgcatcctcacatctacctgatgaggaagttactatcactgccccagcttatagaagaggaaactgaagttcagcagcgtaaatcaatgtacccaaggccaaaaaccagaaatggacatggctggaattccaaattatgtctgcctgactccagaacctgagctcggaaccactctgctctctaaactaacagggaacagctcccaggtcccaacgtaagatagaactctcttctctggccagcctccttcccaacccatcatgcaggctgcgctggaacacatccgttatgtaacagcaccccgaatgaggtcttcttgggctggagggtgtagaggaatcaggacacaggcgcaggctgcctctctgaagcagccaggagagacaggcaaacaggtggcagctggaggcagatgctagtccccaaacagagattggaatggccacttcatttcccttggttcacccttgccccgagatgttagctggcaggaagagaggagggaaggactcgttcaaacagtcaaaacaaggcaggggttcctttctcacacacctcagaaggcaagggtcacacagggcctgggggaaggaagagacaaatctgcttagtccagagtgcttcaacaacagcttactcagaagagtcgaagtggcctcctgccccagccaggccttcacacttcacagcctctgctcatggccaggggcagcccggaagggctggagaaagtaaagagcagacaaggtgagctacctccctggcccaagccatggctctccagggcctcggcagagcccctttccagatgtactcaggacagaaagtacccacccgggccaggagacacccctgaggttcctggtttggggagaggctcccaggggcccctggcagcaccaggagagccaggccgttgattcctggcagagaaggagagtttccagtgacatgtgctttctaaaattagcggcccaggacctcgtggcctagggctcaggtttccctgcctcagcccccagctgcccaccagcctgccccgcactgggctacagcctgaaggtggaggaagctactgagcgccctaggagccagagagaaacaatgcatctgactcacatcggcatggccagaagtcaatggagaggcctagaaagaaaggcaagtctgactaagacccaggcccccggcaaggagctgcccagccccagagcggatcccagtgatgtagaaagaggaagaggaccgctcctcccagctggaattgaggggtgggggtcatgccacctggtggtagagagaggaccaagcaagactgaaggctatactccccgccaccaggccaggcaagcggctgctggtgagtgcccatggctgtcaccccagtacccagggagatagctaacacaaatgcttccgcggcagtgcagcagaggcccagctcttttcggaccgtcccaggcccttcccggctattgagaaccagggcttccaagataggccagggcatacacaaagtccagcgcaagatccacgctgtgtgtgtccgaaagcctggccctgctcagccccagcccaggccttcagttcccagccttgagacagtctggggctcccctctgccaggccccggttccccttcctcttgccaaccctcacaggcgctccccacccccacagcaccccgggcatactcctcccactgcacccccagcccgatagttctttttcacaccttctaggtcctctctcttcctgctggatgacccgggatcattctccccccaggaacctcaccttcaactgcctccttcctggagtcaccctgcccaagcccctggtcttttccctcccatatattcctcaacctaggctggccaaggcctgcccttccaagccagcagcagggccaccagtggcctcctaaccgcccaggccggaggtcaccctgaactccttgctctgctgctaagttaccctcctgaggtcccctcgcaacaccctcctcccactgttattctgctccctctggggtctgcactcttcagctgacaccctataccttcctcccagccactcttatccccgaaagggttttctctgtggcccagactcatacctaacctcctgctaaacattggctcctggatgtccccagagacattctagactcagcttgtccaaaacgggccttcccttgtcctgcctgacctgaccacctcgtgtagcccctgctgtagtggtgggcaggcaaaccgccttggactcagccctcttggcccccagcccaagccacaacccagcactttccatgtcaactgcaaacatgcccaccatcatccccactgctggcgcctcctccctatctgctgccatacggctttcccatccacctcccagagcaaggcaaatccgaccatgtcagccctctgcttaagccacctgctgccagcacgcatgccctcagtgagctctcctccttcactaaccacgtggccccctgctctagtaacacctaacccctcaccattcctggaacacgcctggctctgtgtggcagttctccaggccggaatgtcctctcgacccagctcaatcctcacctccccccagaaacccttttggatctcccaccccatcagagggacgccttctgggggctcctgcagcagccccccaggcacccgcatgtaactacctcattctctgttctctgcgtggctgccatccgtttatatggctgccctaccaggctatgaaggtctttaggctgggcactgtgccttcatctctgcactcccatacctggcacactgaaaaggggtcttccgcccactccagcaagtatagctaaaaaaaaaaagggggggagggcgcggggctgggcttccagatgactggatcccactcccaggagaggaaatgctccctgacaggtgaggggacagatttgaggctgcacgtaaggctggacagaatctccctgggcctagactgcacctgtgttcacctgggagcctggcaccaagaggggcagaggcagacacagagctgctcagtctagcaacagaggagacagaagacaggagtgggaaggcgccgtctcagacccgttctgatgggcaagccaggctcatggctgcagggggaaaaaacattcactgccgcgacctgaaggcacaacccagagctccagcctctgcatcctcacaccctcaacccccacccagggcccaagcaatgcagaccaggtcctctctgatcactggcatttttcagcctgggagccagccttctagaacattttcccgctccctcacactgggtcactcaggcacgttaacgtgcgcttgtctgttcccttgtagcttcccaggcccccaggacagggcacgaacatggcctttagcttctgcctctgctggatctcccaagtagtcttacccgaatcactgttcttagctattcatttccagaaaacaggaaagaacctaagagccaaaggcaactcctacagatacagggtggtcaccaatagaatggcctggggtccaaaaaaaggccagtgaacgaaacttaacagaatccagatgtggccttggaagacacatggcagccccaatgcctcaatctgactgggctttcttgatagaatgttgttggacactgagcagggctatcgtgcttttataaaaggttgagtaaaccagagaaggcaggagaaacagaacctctccacagactagagaaacagggccaaccatatcaaatggagagagccatggctcataagcacttttcagcagccctgtcttcccccatgagcaaggggaagaggacacgggcttaataggaaatggagaaggagcaagtcccgaccaaaagattccatgctgtggccacccccggcccgccctgctgacgggtttcaggcgagtcaagtcattcaacccccagcccctgcatacacatggtgttcacataagctcactcctcagcccccagccggcagaaagccggtgtcccagcgccacctgctgactttccaggcctaccgcagggtggccagtggactctgggtgaacacgccccagctgtggaagaaaaaaaatgaggcagcgcccaggcaaggaagcaagtcaggtgacgcctcaggaaggcttcagtgaagaagaatgactaacaccagggcttccactgccctcagcgactcttacccaccagtctggaatcaggaaaacaggttacaactgggagagtcacctagagcagacccgagaaggctgccccaaagggctgccccaagtccattttggtacagctgcgtggccttccctgtagcctcccagcacacagacgctggagaagacgggaagaggagggctagagctgggggaaatggaggccgtttcaaatgagaacatgacttgtggcagctccagcccacgacccagatggagctcacccatcctgaggacagtgcactaagcgcagggcaaaggggcaggtgtgggtctggcctgtcctcccttcttcttgagaacaagtgacacagaccagctgggtttctggggttttgctgtgtatcttttttaaaaccagctatctgaggggtttggggtaagctggagggtagagagcaaccgactgaggtaagacaacttaggcaaaggtagtctgtgattagatgactcaacctaaaaaagaagaaaaagcagctcagcagagaagcacgggcagctccatctgggctaatggcagcgatgggattctaccctggaggggtaaagaggaaacaaaagatgcctgtggatcaagttcaggtcagcaaaaattcagggggcttccacacaaacaggggccttcctgcgactggctgctaaccagcactttgggcctaaccttgaccgtcatttaagctgagtaaggcagagaaggcagtgcaggtcctctgaacacacaaaccccagcccagagggagctgccgtccccaacacactccaagactcaagagggcctctcgctagctgtgcccccgaagtgcaaggttggcaggaagggaacaggagcgactgccggagtcttccacaagtggaaaccagtggctcatccagtgtggtcccctggaggtggccccgatggacccgccttcacaaactgtcatagctcctaagacctgaaaagctgggcttcttggctaaaaagcccaacaagttcaacccaggcacgcacctaaagctgtcgccgtcagcccgggacagcccattcagtcaccaaatgtttcagcgcccttcatatgtgccaggcccttggcactgagctgaacagtctgaaggggaagagcccaggttttccacgatgggcaaccctgccaagtgccacacctcagagctgcgtgtgcaggctgccctgggacccgaggacagcgctatgggtcagccgggaacatggtgtgggcccctcggaacaggctccacagggaagcctcggagattcacgaagaggaggtgccggctgggccggcagctggagggggtgttccgcacagaggtccccaaaatgctcagagaatcgagttgggggagagcatgtgttacgtgaggctctcccatgagacccacatgactgcttcatgacagggggaggccgaagcagagactgtgggggagccgcgtcctggaggatccatgtgatagcgagccactggaagtggggtgcacaggccaaaggggggaaggcaggtggcagggagcccgcttggtctatacgggatggtggtggccccaccacagcagtggtcccaagggttgtgagagagaggcttaggaggtgacatctacaggctgtttcatgggtggagtccagctctgcaggctgaagacttctggaggttggctacttgacaccgtgaaagcgcctcaccctgctgggccacacactgagaaatggccacgatggttgggcagtcacatgggacaagaagaaagggcagagcagccccaggcttctgggtcaagtgacaggactgagacagtagtggcagaggcaggacaaaagctcagaaggctttggctgggaagctgggactctcccactgctatcccaggcagcagcagcagactatggggggccaagggtacagacttgcttctaggtgtgatgtttcctttcaggccaggccccctttcccaattacaaaggctactcgggagctctcaggctaacctcctatgtgttctgagcccagtcccgctgaaaactagtgccaagcaccaggccttctccagaatgtgctcccctccttggccactaacctgctcacatcctccttcttgatcttgcttccctcttccttctgctccccgatcttctatcgctctgctggaggctggaatccatcctgccagcacattccctttgccctggcctcaatgcctctgaagccagcaacccaagctcgactgcccggaagcaccctatcctgctcatctgccaggcctcccctgctcaaccctgctctccctgtcccctcctttccttgctgcccccaggcctggccagaagtcccactctgcaaccagccctcacacctagcacgatagtgttactccatgggcagccagagctccctttccagcagggggctgcgtcctcgcattccgcaagtccacagcagaaccaagatcatctcagactcccagagactggaaaagcctgctgattcaactccacctgggcctctcagctctgtcccctccaccccacttctactaccactgtaccactgcccccgttcaggttcccagcaagtctcactgacaacctccaacttggtctccccacttcaggctctcctgctccactccaacccatacacccttgcaaaatgttaatccacacaggtgactgcatgccagcagtactggaatacccactaggcaggctctctaccacgcagaaaagttgcatacgaagtctggaacccttaactcctaaccatctaacctgctcgggccatgagtacctgctcgcgccatgagtacctgctcgcgttcaagaactgagcctctcagtgggacataaagaacatggaaagaaagagaggtgggtgtggtggctcatgcctgtattcccagcactttgggaggccgaggtgggcagatcacatgaggccaggagtcagagacgaccagcctggccaacagggcaaaaccgtctctactaaaaatacaaaaattagccaggcgtggtggcatgtgcctgtagtcccagctacttgggaggctgaggcatgagaactgcttgaacccaggaggaggaggctgcagtaagccaagactgtgccactgcactccggcctcagcgacacagagagactctgtctcaaaaaaaaaaaaaaaagaaagaaagaaagaaaaagaaaaagaaaaaaatcaacagcaacaaaaaagaaagagacagataataggagtggcatgggtgctccaagaggatcaggaggcccaaagaaagctgactagctgaggccactgtttatgacatcagaaacagagctgcaggctcgacatccaccaatgaggaattgggtagacactcaagaacactcaagaacgctggagaggccaggcacagtggctcatgcctgtaatcctagcactttgggaggatgaggtgggaggatttcttgagcccagcagtttgagatcagcttgggcaacagagcaagactctgtctctacaaaaaattaaaaaattagcagcacgtggtggcacatgcctatagtcccagctactcgggaggctgaggcaaaagggcggggctgcagtgagccatgatcacaccaatgcactccagcctgggtgatggagcgagaccttgtctcaaaaaaaaaaaataaataaataaataaataaatatgctggaaacaggtcagttgtcccagaaaaacattcatgataaactgagtagaacactcaagtcaccaaggggcatttaaagcatgtggtgcttaaaagccccgtggttaactttttttaaacacgggaatgtttttaaaaagcatgtggaggctgggcgtggtggctcagctgccgcactctcccgtgttcccatccagtagcctgatccaaaaaagccatgaggttggtcttgcgtgacttcttagaaaaggaaacggtgatcccagagatcagtgtggattcaccagttgcccataagcgatctagttaatcatttctggaattttgccagaaatatatactccttgctagtctaagagttaaatctaagatggtggctgtgaccctagaggaccttggctttctgtgtgatgctcttgtccagccttatggccacacagcccatttgcagcttgcaggaaacactgaaaaaacaaagcaggccaggcgcggtggctcacgcctgtgatcccagcactttgggaggctgaggcaggcagatcacctgagctcaggagtttgagaccagcctggccaacatggtgaaaccccgtctctactaaaaatacaaaaaattagcagggcatggtggcgggcgcctgtagtcccagctacttgggaggctgagacaggagaatggcgtgaacccgggagacagagcttgcagtgagccgagatcgagccactgcactcctgcctgggcgacagagtgagactctgtctcaaaaaaaaagagtgagccccctaaggcttctatgtgtatgtgtggatagagccaatccacatggcagccactcatgtgggttaggaccacacctaactacactgttctgccagcaccttccccacacttcggcatctggtcgttaagaacagactcagactggcaagcatcatgctaaaaatccatctacactccatctatagaaggctgacagaacttctccaggaattcacagcagtcaccaatgatcccgctcttcttttccaaaagcatacaagctattctttctagagcatggctggagatcaaagtcaaccctccccaggtcaattcctcatctaccttctcctccctttggaaattgggatgtttgccaggctgcagtctgccagtatctctcccactctgcaaatcctcaagcatcggtggcaatgccttctgcagatgatctcaacagcttctggacataatctcatcaagtacatcctttagggatgccactcttgcccacctgtaatttccattttctcatcctagtgtttcctttgcccttgaccacctaccctccatctccttccttacacagacatataaaagggactgccttctcctcacttctccatgtctttcatgattcagagctcccggcaggttttcaccttcccgatactattcttttttttttttttttttttttttgagatgtcacccaggctggagtgcagtggtgtgatctcggctcactgcaacctctgcctcccgggttcaagcaattctcctgcctcagcctcccgagtagctggcactacaggcacctaccaccaagcccagctaatttttttgtattgttagtagagactgggtgtcaccatgttggccaggctggtctcgaactcctgacctcaggtgatccacctgcctcggcctcccaaagtgctgggattacaggcctaagtcaccgcacccagcccacccctcccccataccattcttaaagctctagatgcttttctggtagatggcctagtttcctctctttccctctcaatgtggtcttttttcctcattttgctgactgtcacactcagaattctgggtttgagactcagtctttcagatctgcttccctttcagcctcagaccacaagataatacttgtttggaacttcctgaaaaatttagggtatgtgtctgactcctcccagccttcctgactttcctaagtttgaagacagcaagcttgtagatcaaatctgtgatcaaacccattatcttgaaaaaaatgtgtttgccttttctagctccacccctctttccaacttggtcgcagagagtaccagatcatctaaacaacagattttaagacaagtagtcatcgtagcgcctagtaaagcaggacacaccaggtgactagagagcaagaatctcctaggcatggagattcttgagtctcggggcacaaaaccaagtggggaataactgtccatgagcctgagaatcacttggtgctatggtctgagtgcccttcaaacttcatgtgttggaatttaatcctcactgccgtagcattaagaggtggggtcttttgagaagtgattaagtgatgagagctccaccctcaaagcaagcgcctttccaatgccttcatacatggtctgagctcccatccacctcccagccaggccctgctgatcagaacggctatgtgaagcaggaggcagcaaacagggccccaggctcaaataggcacttcgtagtggtctagttttgcccgactagttacccttagccttgattaaggtacttagttttaccaaaaaaatcatcagaaatactctggctgccatggaatgtaacatgtcctcattacgagtttcacgtggggaaggccctgaggtgaggagaggcccagcctcttcgtgccacttttacctgctgtccctaggtcaacaccccggacacaaagagtcccccattcagtcgctcccttgtgagctggactctgaaggtcctctcccagaggagggcaaggccttaccgttacatctcactctccatgcaaacagaccgtgagatagtcatctgtttgcctgagagtatgtggtgtgtgagggtcttctgatatttcaggcagccctctcctactctccacgctgcctctggaggtcaggagaaaactatgtggcttccctaacacagacagggctt

SEQ ID NO: 20 - DNA, 510 bp, Homo sapiens
  misc_feature (430)..(430): n=A or C or G or T or U or unknown or other
atttataaatttattgcctgttttattataacaacattatactgtttatggtttaatacatatggttcaaaatgtataatacatcaagtagtacagttttaaaattttatgcttaaaacaagttttgtgtaaaaaatcgcagatacattttacatcggcaaatcaatttttaagtcatcctaaagattgatttttttttgaaatttaaaaacacatttaatttcaatttctctcttatataacctttattactatagcatggtttccactacagtttaacaatgcagcaaaattcccatttcacggtaaattgggttttaagcggcaaggttaaaatgctttgaggatcctgaatacacctttgaacttcaaatgaaggttatggttgttaatttaaccctcatggcataagcagaggcacaagttagcnggcatggtgctctagactggtagagccgagccaccggtgagaagcaanggacagcagcaggaagagccatgggacccccc

SEQ ID NO: 21 - DNA, 60 bp, Homo sapiens
ttaatcctggaaattgtgattgtgacccatgagtggaggaactttcagttctaaagctga

SEQ ID NO: 22 - DNA, 60 bp, Homo sapiens
aagttgtgtagtaaagcattaggagggtcattcttgtcacaaaagtgccactaaaacagc

SEQ ID NO: 23 - DNA, 60 bp, Homo sapiens
aaggccctcttggttttggagagaaagacaagttatgagtagctgctaccctggaacggt

SEQ ID NO: 24 - DNA, 60 bp, Homo sapiens
ggtgggataatcgagtttcagtgacccacgtcagttacacattaaagccagaccccatga

SEQ ID NO: 25 - DNA, 60 bp, Homo sapiens
gtacttaatgttatccagtattgttcattaaatggtgttatcctaaagctgcacttggga

SEQ ID NO: 26 - DNA, 62 bp, Homo sapiens
gaaagcactttgtaggggaactttagtaagttcttctcatttcattatgtttcttccaagga

SEQ ID NO: 27 - DNA, 57 bp, Homo sapiens
tcgtacaatctaccaaccaaccagtgctgaagagattttagaaccttgtaacataca

SEQ ID NO: 28 - DNA, 60 bp, Homo sapiens
ttgtctacgttgaaagcatctgccgtgtagaaacgttatccatgtccggaaagattctgt

SEQ ID NO: 29 - DNA, 57 bp, Homo sapiens
ttcaggtcaccctcaaatcacactctctttaggcaaaacaggaaacttcttaagtga

SEQ ID NO: 30 - DNA, 58 bp, Homo sapiens
aatattagaggatactttgctgtgcacaattccaagtgccttagaacattgtttagct

SEQ ID NO: 31 - DNA, 60 bp, Homo sapiens
agaatattgcctagcccaaatgaacaaagtttagcctaaatctctgtagcatgcaaatca

SEQ ID NO: 32 - DNA, 58 bp, Homo sapiens
aggaaaccttcgaatctgagaacttccacacctgaggcacctgagagaggaactctgt

SEQ ID NO: 33 - DNA, 62 bp, Homo sapiens
tggccccaaatttgctattcccatgcattttgtttgtttcttcacttatcctgttctctgaa

SEQ ID NO: 34 - DNA, 60 bp, Homo sapiens
accacatgcacatccttactacagaatccgtcctttcatttcaacttatagcaagctatg

SEQ ID NO: 35 - DNA, 58 bp, Homo sapiens
ttaactacctcaactggtcagaaacacagattgtattctatgagtcccagaagatgaa

SEQ ID NO: 36 - DNA, 60 bp, Homo sapiens
aggagtatgctgttttcctggcactcatcactgtcatgtgcaatgacttcttccagggct

SEQ ID NO: 37 - DNA, 59 bp, Homo sapiens
gggtaagagtcttgtgttttattcagattgggaaatccattctattttgtgaattggga

SEQ ID NO: 38 - DNA, 60 bp, Homo sapiens
tgcatgtcgtgaccaactagacattctgtcgccttagcatgtttgctgaacaccttgtgg

SEQ ID NO: 39 - DNA, 60 bp, Homo sapiens
atagatctaactttcataggcaaaacaaaagcttcgagctgttgcgtgtgtgagtctgtt

SEQ ID NO: 40 - DNA, 56 bp, Homo sapiens
aggtgtattcaggatcctcaaagcattttaaccttgccgcttaaaacccaatttac

(19) United States
(12) Patent Application Publication
(10) Pub. No.: US 2004/0241725 A1
(43) Pub. Date: Dec. 2, 2004

(54) Lung cancer detection

(76) Inventors: Wenming Xiao, Gaithersburg, MD (US); Gang Dong, North Potomac, MD (US); Philip Reena, North Potomac, MD (US)

Correspondence Address: BLANK ROME LLP, 600 NEW HAMPSHIRE AVENUE, N.W., WASHINGTON, DC, 20037, US

(21) Appl. No.: 10/807,308

(22) Filed: Mar. 24, 2004

Related U.S. Application Data
(60) Provisional application No. 60/456,938, filed on Mar. 25, 2003.

(51) Int. Cl. C12Q 1/68; C07H 21/04; C12M 1/34

(52) U.S. Cl. 435/6; 435/287.2; 536/24.3

(57) ABSTRACT

The present invention relates to a method, apparatus, polynucleotide markers and its related products for detecting non-small cell lung cancer (NSCLC). Particularly, the method and apparatus of the present invention can detect and differentiate between adenocarcinoma, squamous cell carcinoma, and normal lung tissues. Twenty markers for NSCLC are disclosed. By probing for at least 6 of the 20 genes, detection of NSCLC cancer can be detected with at least about 90% accuracy.

### FIELD OF THE INVENTION

The present invention relates to a method, apparatus, polynucleotide markers and related products for detecting non-small cell lung cancer (NSCLC). Particularly, the method, apparatus and products of the present invention can detect and differentiate between adenocarcinoma, squamous cell carcinoma, and normal lung tissues.

### BACKGROUND OF THE INVENTION

Lung cancer is the primary cause of cancer death among both men and women in the U.S., with an estimated 156,000 new cases being reported in 2001 (Minna et al. (2002), Ann. Rev. Physiol., 64: 681-708). The five-year survival rate among all lung cancer patients, regardless of the stage of disease at diagnosis, is only 14%. This contrasts with a five-year survival rate of 46% among cases detected while the disease is still localized. However, only 16% of lung cancers are discovered before the disease has spread.

Early stage lung cancer can be detected by chest radiograph and the sputum cytological examination; however, these procedures do not have sufficient sensitivity for routine use as screening tests for asymptomatic individuals. Potential technical problems which can limit the sensitivity of chest radiograph include suboptimal technique, insufficient exposure, and positioning and cooperation of the patient (T. G. Tape et al. (1986), Ann. Intern. Med., 104: 663-670). Moreover, radiologists often disagree on interpretations of chest radiographs; over 40% of these disagreements are significant or potentially significant, with false-negative interpretations being the cause of most errors (P. G. Herman et al. (1975), Chest, 68: 278-282). Inconclusive results require additional follow-up testing for clarification (T. G. Tape et al., supra).

Sputum cytology is even less sensitive than chest radiography in detecting early lung cancer. Factors affecting the ability of sputum cytological examination to diagnose lung cancer include the ability of the patient to produce sufficient sputum, the size of the tumor, the proximity of the tumor to major airways, the histological type of the tumor, and the experience and training of the cytopathologist (R. J. Ginsberg et al. (1993), In: Cancer: Principles and Practice of Oncology, Fourth Edition, V. T. DeVita, S. Hellman, S. A. Rosenburg, pp. 673-723, Philadelphia, Pa.: J. B. Lippincott Co.).

Attempts have been made to discover improved tumor markers for lung cancer by first identifying differentially expressed cellular components in lung tumor tissue compared to normal lung tissue. The tumor markers can be an antigen or a polynucleotide. With a protein, detection usually requires an immunoassay using monoclonal antibodies (MAbs). MAbs for lung cancer were first developed to distinguish non-small cell lung cancer (NSCLC) from small cell lung cancer (SCLC). (Mulshine, et al. (1983), J. Immunol., 121:497-502). In most cases, the identity of the cell surface antigen with which a particular antibody reacts is not known, or has not been well characterized. (Scott, et al. (1993), “Early lung cancer detection using monoclonal antibodies,” In: Lung Cancer. Edited by J. A. Roth, J. D. Cox, and W. K. Hong. Boston: Blackwell Scientific Publications).

MAbs have been used in the immunocytochemical staining of sputum samples to predict the progression of lung cancer (Tockman, et al. (1988), J. Clin. Oncol., 6:1685-1693). In the study, two MAbs were utilized, 624H12 which binds a glycolipid antigen expressed in SCLC and 703D4 which is directed to a protein antigen of NSCLC. Of the sputum specimens from participants who progressed to lung cancer, two-thirds showed positive reactivity with either the SCLC or the NSCLC MAb. In contrast, of those that did not progress to lung cancer, 35 of 40 did not react with the SCLC or NSCLC Mab. This study suggests the need for the development of additional early detection targets to discover the onset of malignancy at the earliest possible stage.

Despite the numerous examples of MAb applications, none has yet emerged that has changed clinical practice (Mulshine, et al. (1991), “Applications of monoclonal antibodies in the treatment of solid tumors,” In: Biologic Therapy of Cancer. Edited by V. T. Devita, S. Hellman, and S. A. Rosenberg. Philadelphia: J B Lippincott, pp. 563-588). MAbs alone may not be the answer to early detection because there has only been moderate success with immunologic reagents for paraffin-embedded tissue. Secondly, lung cancer may express features that cannot be differentiated by antibodies directly; for example, chromosomal deletions, gene amplification, or translocation and alteration in enzymatic activity.

A more recent approach is to screen for polynucleotide markers of lung cancer. U.S. Pat. No. 6,316,213 to O'Brian discloses a method for early diagnosis of ovarian, breast or lung cancer by screening for PUMP-1 mRNA or PUMP-1 protease. The diagnosis can be accomplished by an immunoassay to detect the PUMP-1 protease or a hybridization assay to detect the PUMP-1 mRNA.

U.S. Pat. Nos. 5,589,579 and 5,773,579, both to Torczynski et al., disclose a polynucleotide marker (HCAVIII) for NSCLC and its corresponding amino acid sequence. Hybridization assay and immunoassay for the marker is also disclosed for the detection of lung cancer.

U.S. Pat. Nos. 6,251,586 and 5,994,062, both to Mulshine et al., disclose an epithelial protein and corresponding DNA for use in early cancer detection. The protein is purified from two human cancer cell lines, NCI-H720 and NCI-H157. Methods for monitoring the expression of the epithelial protein and mRNA are disclosed as a screen for lung cancer.

Other patents disclosing markers (polynucleotides and/or polypeptides) for lung cancer includes U.S. Pat. Nos. 6,312,695 and 6,210,883, both to Reed et al.; U.S. Pat. No. 5,939,265 to Cohen et al.; U.S. Pat. No. 5,935,786 to Nakamura et al.; and U.S. Pat. No. 5,670,314 to Chrisman et al.

The problem with the efforts to date in the detection and diagnosis of lung cancer is that they are based on the measurement of a single gene/molecule which measurements are subject to unpredictable reliability and accuracy due to the skills required in running the assays.

Classification of human lung cancer by gene expression profiling has been described in several recent publications (M. Garber, “Diversity of gene expression in adenocarcinoma of the lung,” PNAS, 98(24): 13784-13789 (2001); A. Bhattacharjee, “Classification of human lung carcinomas by mRNA expression profiling reveals distinct adenocarcinoma subclasses,” PNAS, 98(24):13790-13795 (2001)), but no specific gene set is used as a classifier to diagnose lung cancer in unknown tissue samples.

Large gene sets containing on the order of from 75 to 100 sequences or as many as 50,000 to 60,000 sequences may be used as a research and diagnostic tool, however, the need exists for a smaller, more concise gene group for use in the detection and differentiation of lung cancer. In particular, the smaller gene set and associated products are far more amenable to a kit format and for the generation and interpretation of recognizable patterns which are the basis of the present invention.

### SUMMARY OF THE INVENTION

The present invention provides a set of polynucleotides as marker for NSCLC, including adenocarcinoma and squamous cell carcinoma. The set of polynucleotides comprises about 6 to about 20 sequences selected from the group consisting of SEQ ID NOS: 1-20.

The present invention further provides a gene chip for the detection of NSCLC. The chip comprises probes for specifically binding with about 6 to about 20 sequences selected from the group consisting of SEQ ID NOS: 1-20. Preferably, the probes are selected from the group consisting of SEQ ID NOS: 21-40.

The present invention further provides methods for detecting NSCLC. The methods comprise contacting a tissue sample with probes that specifically bind with about 6 to about 20 gene products selected from the group consisting of gene products of SEQ ID NOS: 1-20, and correlating the binding pattern with the presence or absence of NSCLC. Preferably, the probes are selected from the group consisting of SEQ ID NOS: 21-40.

The present invention further provides methods for distinguishing between adenocarcinoma, squamous cell carcinoma, and normal tissues. The methods comprise contacting a tissue sample with probes that specifically bind with about 6 to about 20 gene products selected from the group consisting of gene products of SEQ ID NOS: 1-20, and correlating the binding pattern with adenocarcinoma, squamous cell carcinoma, or normal tissues. Preferably, the probes are selected from the group consisting of SEQ ID NOS: 21-40.

The present invention further provides methods for monitoring the treatment of a patient with lung cancer. The methods comprise administering a pharmaceutical composition to the patient, obtaining a tissue sample from the patient, contacting the tissue sample with probes that specifically bind with about 6 to about 20 gene products selected from the group consisting of gene products of SEQ ID NOS: 1-20, and correlating the binding pattern with the effectiveness of the pharmaceutical composition in treating lung cancer. Preferably, the probes are selected from the group consisting of SEQ ID NOS: 21-40.

The present invention further provides methods for screening for an agent capable of modulating the onset or progression of lung cancer. The methods comprise exposing a cell to the agent, extracting a gene product sample from the cell, contacting the gene product sample with probes that specifically bind with about 6 to about 20 gene products selected from the group consisting of gene products of SEQ ID NOS: 1-20, and correlating the binding pattern with the effectiveness of the agent in modulating the onset dr progression of lung cancer. Preferably, the probes are selected from the group consisting of SEQ ID NOS: 21-40.

In embodiments of the invention, the isolated gene set has less than about 400 sequences comprising from about 6 to about 20 sequences selected from the group consisting of SEQ ID NOS: 1-20. In other embodiments of the invention, the probes that specifically bind to from about 6 to about 20 sequences selected from the group consisting of SEQ ID NOS: 1-20 are greater than about 30 nucleotides in length.

In embodiments of the invention, the hybridization of the sample with the probes generates an expression pattern. The expression pattern may be used in the methods of the invention for a variety of uses as described herein, for example, for the comparison of the expression pattern of a healthy individual with the expression pattern of a diseased individual.

The gene products as recited herein can be DNA, RNA, and/or proteins. In the case of DNA and RNA, binding occurs through hibridization with oligonucletide probes. In the case of proteins, binding occurs though various protein interaction; and the probes can be but are not limited to enzymes, antibodies, cell surface receptors, secreted proteins, receptor ligands, immunoliposomes, immunotoxins, cytosolic proteins, nuclear proteins, and functional motifs thereof. Because the gene products can be in the form of diffusible factors present in the patient's serum, the present invention can also be used to develop a non-invasive blood test for lung cancer.

### BRIEF DESCRIPTION OF THE DRAWINGS

FIG. 1 shows a flow chart of the selection process for the marker genes and fragments for lung cancer.

FIG. 2 shows ANOVA result for the 20 selected genes and fragments when compared to house keeping genes.

FIG. 3 shows the PCA plot and separation of NSCLC for the 20 selected genes and fragments (SEQ ID NOS: 1-20).

FIG. 4 shows the PCA plot for 72 house keeping genes.

FIG. 5 shows the effect of smoking status on the assay's ability to differentiate between different types of NSCLC.

FIG. 6 shows the effect of sex on the assay's ability to differentiate between different types of NSCLC.

FIG. 7 shows the effect of race on the assay's ability to differentiate between different types of NSCLC.

FIG. 8 shows the effect of medication status on the assay's ability to differentiate between different types of NSCLC.

FIG. 9 shows the relative expression levels for normal and NSCLC samples.

### DETAILED DESCRIPTION OF THE PRESENT INVENTION

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of groups of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells (Marshall, (1991) Cell, 64, 313-326; Weirlberg, (1991) Science, 254, 1138-1146). Thus, changes in the expression levels of particular genes (e.g., oncogenes or tumor suppressors) serve as signposts for the presence and progression of various diseases.

Monitoring changes in gene expression may also provide certain advantages during drug screening development. Often drugs are screened and prescreened for the ability to interact with a major target without regard to other effects the drugs have on cells. Often such other effects cause toxicity in the whole animal, which prevent the development and use of the potential drug.

The present inventors have examined tissue samples from normal lung, adenocarcinoma, and squamous cell carcinoma to identify a gene set associated with lung cancer. Changes in gene expression, also referred to as expression profiles or expression pattern, provide useful markers for diagnostic uses as well as markers that can be used to monitor disease states, disease progression, drug toxicity, drug efficacy and drug metabolism.

Uses for the Lung Cancer Markers as Diagnostics

As described herein, the genes of SEQ ID NOS: 1-20 may be used as diagnostic markers for the prediction or identification of lung cancer. For instance, a lung tissue sample or other sample from a patient may be assayed by any of the methods described herein or by any other method known to those skilled in the art, and the expression levels from a gene or genes from the SEQ ID NOS: 1-20 may be compared to the expression levels found in normal lung tissue. Expression profiles generated from the tissue or other sample that substantially resemble an expression profile from normal or diseased lung tissue may be used, for instance, to aid in disease diagnosis. Comparison of the expression data, as well as available sequence or other information may be done by researcher or diagnostician or may be done with the aid of a computer and databases.

Use of the Lung Cancer Markers for Monitoring Disease Progression

As described above, the genes and gene expression information of SEQ ID NOS: 1-20 may also be used as markers for the monitoring of disease progression, for instance, the development of lung cancer. For instance, a lung tissue sample or other sample from a patient may be assayed by any of the methods described above, and the expression levels in the sample from a gene or genes from SEQ ID NOS: 1-20 may be compared to the expression levels found in normal lung tissue, adenocarcinoma tissue, or squamous cell carcinoma tissue. The gene expression pattern can be monitored over time to track progression of the disease. Comparison of the expression pattern, as well as available sequence or other information may be done by researcher or diagnostician or may be done with the aid of a computer and databases.

Use of the Lung Cancer Markers for Drug Screening

According to the present invention, the genes identified in SEQ ID NOS: 1-20 may be used as markers to evaluate the effects of a candidate drug or agent on a cell, particularly a cell undergoing malignant transformation, for instance, a lung cancer cell or tissue sample.

Alternatively, a patient can be treated with a drug candidate and the progression of lung cancer is monitored over time. This method comprises treating the patient with an agent, obtaining a tissue sample from the patient, extracting a gene product sample from the tissue sample, contacting the gene product sample with probes which specifically bind with gene products of SEQ ID NOS: 1-20, and comparing the binding pattern over time to determine the effect of the agent on the progression of lung cancer.

A candidate drug or agent can be screened for the ability to stimulate the transcription or expression of a given marker or markers (drug targets) or to down-regulate or counteract the transcription or expression of a marker or markers. According to the present invention, one can also compare the specificity of drugs' effects by looking at the number of markers affected by different drugs and comparing them. More specific drugs will affect fewer transcriptional targets. Similar sets of markers identified for two drugs indicate similar effects.

The agents of the present invention can be, as examples, peptides, small molecules, vitamin derivatives, as well as carbohydrates. Dominant negative proteins, DNA encoding these proteins, antibodies to these proteins, peptide fragments of these proteins or mimics of these proteins may be introduced into cells to affect function. “Mimic” as used herein refers to the modification of a region or several regions of a peptide molecule to provide a structure chemically different from the parent peptide but topographically and functionally similar to the parent peptide (see Grant (1995), in Molecular Biology and Biotechnology, Meyers (editor) VCH Publishers). A skilled artisan can readily recognize that there is no limit as to the structural nature of the agents of the present invention.

Assay Formats

The genes identified as being differentially expressed in lung cancer may be used in a variety of nucleic acid detection assays to detect or quantify the expression level of a gene or multiple genes in a given sample. Any hybridization assay format may be used, including solution-based and solid support-based assay formats, for example, traditional Northern blotting. Other suitable assay formats that may be used for detecting gene expression levels include, but are not limited to, nuclease protection, RT-PCR and differential display methods. These methods are useful for some embodiments of the invention; however, methods and assays of the invention are most efficiently designed with array or chip hybridization-based methods for detecting the expression of a large number of genes. Assays and methods of the invention may utilize available formats to simultaneously screen from at least about 6 to about 100, preferably about 1000, more preferably about 10,000 and most preferably about 1,000,000 or more different nucleic acid hybridizations.

Assays to monitor the expression of a marker or markers of SEQ ID NOS: 1-20 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to at least two genes selected from SEQ ID NOS: 1-20 may be used to directly monitor or detect changes in gene expression in the treated or exposed cell. High density gene chips and their uses are described in U.S. Pat. No. 6,040,138 to Lockhart et al., which is incorporated herein by reference. An alternative format to the gene chip is the flow-through chip disclosed in U.S. Pat. No. 5,843,767 to Beattie, which is incorporated herein by reference.

In another format, cell lines that contain reporter gene fusions between the open reading frame and/or the 3′ or 5′ regulatory regions of a gene selected from SEQ ID NOS: 1-20 and any assayable fusion partner may be prepared. Numerous assayable fusion partners are known and readily available including the firefly luciferase gene and the gene encoding chloramphenicol acetyltransferase (Alain et al. (1990), Anal. Biochem., 188: 245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of one or more genes identified in SEQ ID NOS: 1-20. For instance, as described above, mRNA expression may be monitored directly by hybridization of probes to the nucleic acids of SEQ ID NOS: 1-20. Cell lines are exposed to the agent to be tested under appropriate conditions and time and total RNA or mRNA is isolated by standard procedures such those disclosed in Sambrook et al. (1989), Molecular Cloning—A Laboratory Manual, Cold Spring Harbor Laboratory Press.

In another assay format, cells or cell lines are first identified which express the gene products of the invention physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Such cell lines may be, but are not required to be, derived from lung tissue. Further, such cells or cell lines may be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag. Such a process is well known in the art (see Sambrook et al., (1989) Molecular Cloning—A Laboratory Manual, Cold Spring Harbor Laboratory Press).

Cells or cell lines transduced or transfected as outlined above are then contacted with agents under appropriate conditions. For example, the agent comprises a pharmaceutically acceptable excipient and is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and serum incubated at 37° C. Said conditions may be modulated as necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells; and an increase or decrease in the immunologically generated signal from the “agent-contacted” sample compared to the control will be used to distinguish the effectiveness of the agent.

Another embodiment of the present invention provides methods for identifying agents that modulate the levels, concentration or at least one activity of a protein(s) encoded by the genes of SEQ ID NOS: 1-20. Such methods or assays may utilize any means of monitoring or detecting the desired activity.

In one format, the relative amounts of a protein of the invention between a cell population that has been exposed to the agent to be tested compared to an un-exposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with probes, such as specific antibodies.

The genes which are assayed according to the present invention are typically in the form of mRNA or reverse transcribed mRNA. The genes may be cloned or not and the genes may be amplified or not. The cloning itself does not appear to bias the representation of genes within a population. However, it may be preferable to use polyA+ RNA as a source, as it can be used with less processing steps.

Probes based on the sequences of the genes described herein may be prepared by any commonly available method. Oligonucleotide probes for assaying the tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases longer probes of at least 30, 40, 50, 60 or 70 nucleotides will be desirable. It is preferable that more than one probes specific for each gene are used in the assay.

In a preferred embodiment, a FLOW-THRU® chip, such as that disclosed in U.S. Pat. No. 5,843,767, which disclosure in incorporated herein by reference in its entirety, is used with present invention. The FLOW-THRU® chip generally comprises an array of micro-channels extending through a solid support. Each micro-channel contains a probe specific for a gene selected from SEQ ID NOS: 1-20; and different channels contain different probes for different genes. The hybridization and/or binding reactions take place by providing fluidic flow through of the sample through the chip.

In another embodiment of the present invention, protein and tissue arrays can also be used. In protein arrays, the probes are specific for protein products of the genes of SEQ ID NOS: 1-20. These probes can be, but are not limited to, antibodies, cell surface receptors, secreted proteins, receptor ligands, immunoliposomes, immunotoxins, cytosolic proteins, nuclear proteins, and functional motifs thereof that specifically bind to the protein products of the genes of SEQ ID NOS: 1-20. The probes are immobilized on a solid support to form an array. The supports can be either plates (glass, plastics, or silicon) or membranes made of nitrocellulose, nylon, or polyvinylidene difluoride (PVDF).

To use a protein array in studying protein expression patterns, an antibody array is incubated with a protein sample prepared under the conditions that native protein—protein interactions are minimized. After incubation, unbound or non-specific binding proteins can be removed with several washes. Proteins specifically bound to their respective antibodies on the array are then detected. Because the antibodies are immobilized in a predetermined order, the identity of the protein captured at each position is therefore known. Measurement of protein amount at all positions on the array thus reflects the protein expression pattern in the sample.

The quantities of the proteins trapped on the array can be measured in several ways. First, the proteins in the samples can be metabolically labeled with radioactive isotopes (S-35 for total proteins and P-32 for phosphorylated proteins). The amount of labeled proteins bound to each antibody on an array can be quantified by autoradiography and densitometry. Second, the protein sample can also be labeled by biotinylation in vitro. Biotinylated proteins trapped on the array will then be detected by avidin or streptavidin which strongly binds biotin. If avidin is conjugated with horseradish peroxidase or alkaline phosphatase, the captured protein can be visualized by enhanced chemical luminescence. The amount of proteins bound to each antibody represents the level of the specific protein in the sample. If a specific group of proteins are interested, they can be detected by agents which specifically recognize them. Other methods, like immunochemical staining, surface plasmon resonance, matrix-assisted laser desorption/ionization-time of flight, can also be used to detect the captured proteins.

Tissue arrays consist of regular arrays of cores of embedded biological tissue arranged in a sectionable block typically made of the same embedding material used originally for the tissue in the cores. The new blocks may be sectioned by traditional means (microtomes etc.) to create multiple nearly identical sections each containing dozens, hundreds or even over a thousand different tissue types.

In tissue array, the tissue sample is assayed for differential expression of the protein products of the genes of SEQ ID NOS: 1-20. When analyzing the intracellular localization of a target protein, standard cytoimmunostaining techniques known to skilled artisans can be employed. Cytoimmunostaining may be performed directly on frozen sections of cells or tissues or, preceded by fixing cells with a fixative that preserves the intracellular structures, followed by permeablization of the cell to ensure free access of the probes. The step of permeablization can be omitted when examining cell-surface antigens. After incubating the cell preparations with a probe such as an antibody specific for the target, unbound antibody is removed by washing, and the bound antibody is detected either directly (if the primary antibody is labeled) or, more commonly, indirectly visualized using a labeled secondary antibody. In localizing a target polypeptide to a specific subcellular structure in a cell, co-staining with one or more marker antibodies specific for antigens differentially present in such structure is preferably performed. A battery of organelle specific antibodies is available in the art. Non-limiting examples include plasma membrane specific antibodies reactive with cell surface receptor Her2, endoplasmic reticulum (ER) specific antibodies directed to the ER resident protein Bip, Gogli specific antibody α-adaptin, and cytokeratin specific antibodies which will differentiate cytokeratins from different cell types (e.g. between epithelial and stromal cells) or in different species. To detect and quantify the immunospecific binding, digital image analysis system coupled to conventional or confocal microscopy can be employed.

Probe Design

One of skill in the art will appreciate that an enormous number of array designs are suitable for the practice of this invention. The high density array will typically include a number of probes that specifically hybridize to the sequences of interest. Methods of producing probes for a given gene or genes are disclosed in WO 99/32660, which is incorporated herein by reference. In addition, in a preferred embodiment, the array will include one or more control probes. High density array chips of the invention include “test probes.” Test probes may be oligonucleotides that range from about 5 to about 500 or about 10 to about 100 nucleotides, more preferably from about 20 to about 80 nucleotides and most preferably from about 50 to about 70 nucleotides in length. In other particularly preferred embodiments the probes are about 20 to about 25 nucleotides in length. In another preferred embodiment, test probes are double or single strand DNA sequences. DNA sequences are isolated or cloned from natural sources or amplified from natural sources using natural nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization controls can also be selected to reflect the (average) base composition of the other probes in the array, however in a preferred embodiment, only one or a few probes are used and they are selected such that they hybridize well (i.e., no secondary structure) and have minimal cross match with non-specific targets.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to the 3-actin gene, the transferrin receptor gene, the GAPDH gene, and the like.

Mismatch controls are generally not required when using probes of about 60 to about 70 nucleotides. However, when using shorter probes, mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a twenty-mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe provides a good measure of the concentration of the hybridized material.

However, when using the preferred embodiment of about 60-mer to about 70 mer probes, mismatch probes are not required as the probes are sufficiently long that a single mismatch does not effect an appreciable difference in binding efficiency.

Nucleic Acid Samples

As is apparent to one of ordinary skill in the art, nucleic acid samples used in the methods and assays of the invention may be prepared by any available method or process. Methods of isolating total RNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I—Theory and Nucleic Acid Preparation, Tijssen, (1993) (editor) Elsevier Press. Such samples include RNA samples, but also include cDNA synthesized from a mRNA sample isolated from a cell or tissue of interest. Such samples also include DNA amplified from the cDNA, and an RNA transcribed from the amplified DNA. One of skill in the art would appreciate that it is desirable to inhibit or destroy RNase present in homogenates before homogenates can be used.

Biological samples may be of any biological tissue or fluid or cells from any organism as well as cells raised in vitro, such as cell lines and tissue culture cells. Frequently the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

Solid Supports

Solid supports containing oligonucleotide probes for differentially expressed genes of the invention can be filters, polyvinyl chloride dishes, silicon or glass based chips, etc. Such wafers and hybridization methods are widely available, for example, those disclosed by U.S. Pat. No. 6,040,138 to Lockhart et al. and U.S. Pat. No. 5,843,767 to Beattie. Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, about 2, 10, 100, 1000 to 10,000; 100,000 or 400,000 of such features on a single solid support. The solid support, or the area within which the probes are attached may be on the order of a square centimeter.

Oligonucleotide probe arrays for expression monitoring can be made and used according to any techniques known in the art (see for example, Lockhart et al. (1996), Nat. Biotechnol., 14: 1675-1680; McGall et al. (1996), PNAS USA, 93:13555-13460). Such probe arrays may contain at least two or more oligonucleotides that are complementary to or hybridize to two or more of the genes described herein. Such arrays may also contain oligonucleotides that are complementary or hybridize to at least about 2, 3, 4, 5, 6, 7, 8, 9, 10, 20, 30, 50, 70, 100 or more the genes described herein.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (U.S. Pat. No. 5,143,854 to Pirrung et al.; U.S. Pat. No. 5,800,992 to Fodor et al.; U.S. Pat. No. 5,837,832 to Chee et al; which are incorporated herein by reference).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences has been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in U.S. Pat. No. 5,677,195 to Winkler et al., which is incorporated herein by reference. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing (see U.S. Pat. No. 6,333,155 to Lockhart et al, which is incorporated herein by reference). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids.

Under low stringency conditions (e.g., low temperature and/or high salt) hybrid duplexes (e.g., DNA-DNA, RNA-RNA or RNA-DNA) will form even where the annealed sequences are not perfectly complementary.

Thus specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPE-T at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art (see U.S. Pat. No. 6,333,155 to Lockhart et al, which is incorporated herein by reference). Commonly employed labels include, but are not limited to, biotin, fluorescent molecules, radioactive molecules, chromogenic substrates, chemiluminescent labels, enzymes, and the like. The methods for biotinylating nucleic acids are well known in the art, as are methods for introducing fluorescent molecules and radioactive molecules into oligonucleotides and nucleotides.

When biotin is employed, it is detected by avidin, streptavidin or the like, which is conjugated to a detectable marker, such as an enzyme (e.g., horseradish peroxidase) or radioactive label (e.g., ³²P, ³⁵S, ³³P). Enzyme conjugates are commercially available from, for example, Vector Laboratories, Burlingame, Calif. Steptavidin binds with high affinity to biotin, unbound stretavidin is washed away, and the presence of horseradish peroxidase enzyme is then detected using a substrate in the presence of peroxide and appropriate buffers. The binding reaction may be detected using a microscope equipped with a visible light source and a CCD camera (Princeton Instruments, Princeton, N.J.).

Detection methods are well known for fluorescent, radioactive, chemiluminescent, chromogenic labels, as well as other commonly used labels. Briefly, fluorescent labels can be identified and quantified most directly by their absorption and fluorescence emission wavelengths and intensity. A microscope/camera setup using a light source of the appropriate wavelength is a convenient means for detecting fluorescent label. Radioactive labels may be visualized by standard autoradiography, phosphor image analysis or CCD detector. Other detection systems are available and known in the art.

Databases

The present invention includes relational databases containing sequence information, for instance for the genes of SEQ ID NOS: 1-20, as well as gene expression information in various lung tissue samples. Databases may also contain information associated with a given sequence or tissue sample such as descriptive information about the gene associated with the sequence information, or descriptive information concerning the clinical status of the tissue sample, or the patient from which the sample was derived. The database may be designed to include different parts, for instance a sequences database and a gene expression database.

Methods for the configuration and construction of such databases are widely available, for instance in U.S. Pat. No. 5,953,727 to Akerblom et al., which is herein incorporated by reference.

The databases of the invention may be linked to an outside or external database. In a preferred embodiment, the external database is GenBank and the associated databases maintained by the National Center for Biotechnology Information (NCBl).

Any appropriate computer platform may be used to perform the necessary comparisons between sequence information, gene expression information and any other information in the database or provided as an input. For example, a large number of computer workstations are available from a variety of manufacturers, such has those available from Silicon Graphics. Client-server environments, database servers and networks are also widely available and appropriate platforms for the databases of the invention.

The databases of the invention may be used to produce, among other things, electronic Northerns to allow the user to determine the cell type or tissue in which a given gene is expressed and to allow determination of the abundance or expression level of a given gene in a particular tissue or cell.

The databases of the invention may also be used to present information identifying the expression level in a tissue or cell of a set of genes comprising at least one gene in SEQ ID NOS: 1-20 comprising the step of comparing the expression level of at least one gene in Tables 3-9 in the tissue to the level of expression of the gene in the database. Such methods may be used to predict the physiological state of a given tissue by comparing the level of expression of a gene or genes in SEQ ID NOS: 1-20 from a sample to the expression levels found in tissue from normal lung, adenocarcinoma, or squamous cell carcinoma. Such methods may also be used in the drug or agent screening assays as described above.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### EXAMPLE 1

### Gene Selection for 20 Genes

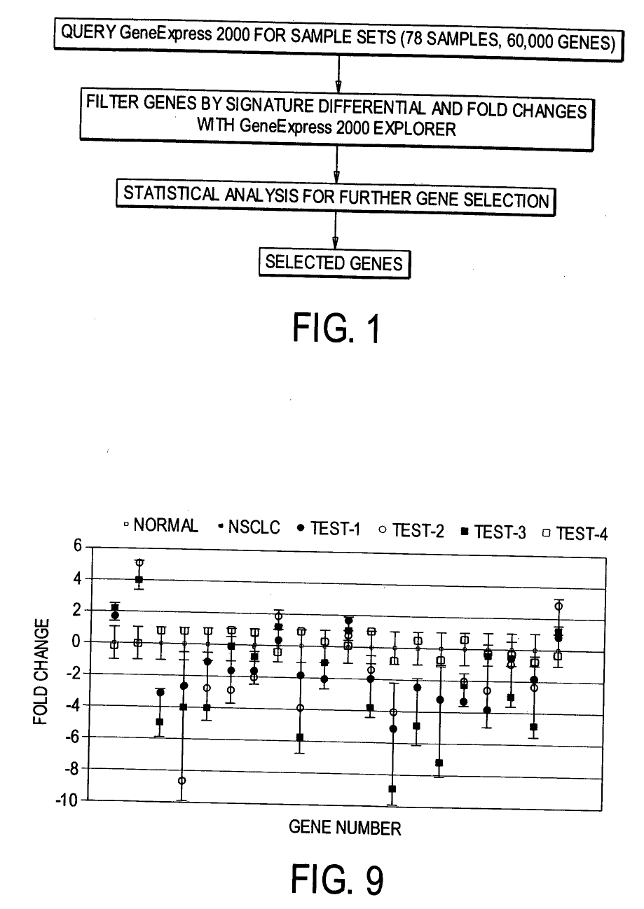

FIG. 1 shows a flow chart of the selection process. From 78 samples available for NSCLC study, expression of about 60,000 genes and fragments were measured with Affymetrix gene chip and stored on GeneExpress 2000®. The 60,000 genes and fragments are then filtered with Gene Signature tool (threshold setting at 95% for both absent and present calls) and Fold Change Analysis tool provided by GeneExpress 2000®.

The expression raw data for the initially selected genes and fragments, in group samples, were exported from the database and further analyzed with Partek Pro 2000®. These genes were subjected to selection with Variable Selection, a tool of Partek Pro 2000®. For the settings of variable selection, linear discriminate analysis was used as the classification model, forward selection was used as the search method and posterior error was used as the modeling error criteria.

The final set of genes and fragments was selected with the perfect score after many iterations. Table 1 lists the GenBank accession numbers, gene symbol (if known), gene name (if known), and UniGene cluster identifiers for the final set of genes and fragments.

TABLE 1

| GenBank | Gene | | UniGene | SEQ ID |
|---|---|---|---|---|
| Acc. No. | Symbol | Gene Name | Cluster Id. | NO: |
| U97105 | DPYSL2 | dihydropyrimidinase-like 2 | Hs.401072 | 1 |
| AI525592 | PIGPC | p53 induced protein PIGPC1 | Hs.303125 | 2 |
| BC009753 | | | Hs.234898 | 3 |
| AL117561 | | | Hs.180372 | 4 |
| BGOI 1189 | | | Hs.301664 | 5 |
| NM_024513 | FYCO1 | FYVE and coiled-coil domain | Hs.257267 | 6 |
| | | containing 1 | | |
| AB018339 | SYNE-1 | synaptic nuclei expressed gene 1b | Hs.8182 | 7 |
| BC011706 | MGC19780 | Hypothetical protein MGC19780 | Hs.124005 | 8 |
| AA524029 | X123 | Friedreich ataxia region gene X123 | Hs.77889 | 9 |
| AI472209 | | | Hs.323117 | 10 |
| T90693 | FLJ22029 | hypothetical protein FLJ22029 | Hs.196094 | 11 |
| AA193416 | SLC27A3 | Solute carrier family 27 (fatty acid | Hs.109274 | 12 |
| | | transporter), member 3 | | |
| AI983204 | ALOX5AP | arachidonate 5-lipoxygenase-activating | Hs.100194 | 13 |
| | | protein | | |
| AL037969 | PPAP2B | phosphatidic acid phosphatase type 2B | Hs.173717 | 14 |
| X14420 | COL3A1 | collagen, type III, alpha 1 (Ehlers- | Hs.119571 | 15 |
| | | Danlos syndrome type IV, autosomal | | |
| | | dominant) | | |
| AI539439 | S100A2 | S100 calcium-binding protein A2 | Hs.38991 | 16 |
| M77481 | MAGEA1 | melanoma antigen. family A, 1 (directs | Hs.72879 | 17 |
| | | expression of antigen MZ2-E) | | |
| U83661 | ABCC5 | ATP-binding cassette, sub-family C | Hs.108660 | 18 |
| | | (CFTR/MRP), member 5 | | |
| U36341 | SLC6A8 | solute carrier family 6 (neurotransmitter | Hs.187958 | 19 |
| | | transporter, creatine), member 8 | | |
| W68630 | | | Hs.161566 | 20 |

### EXAMPLE 2

### ANOVA Test

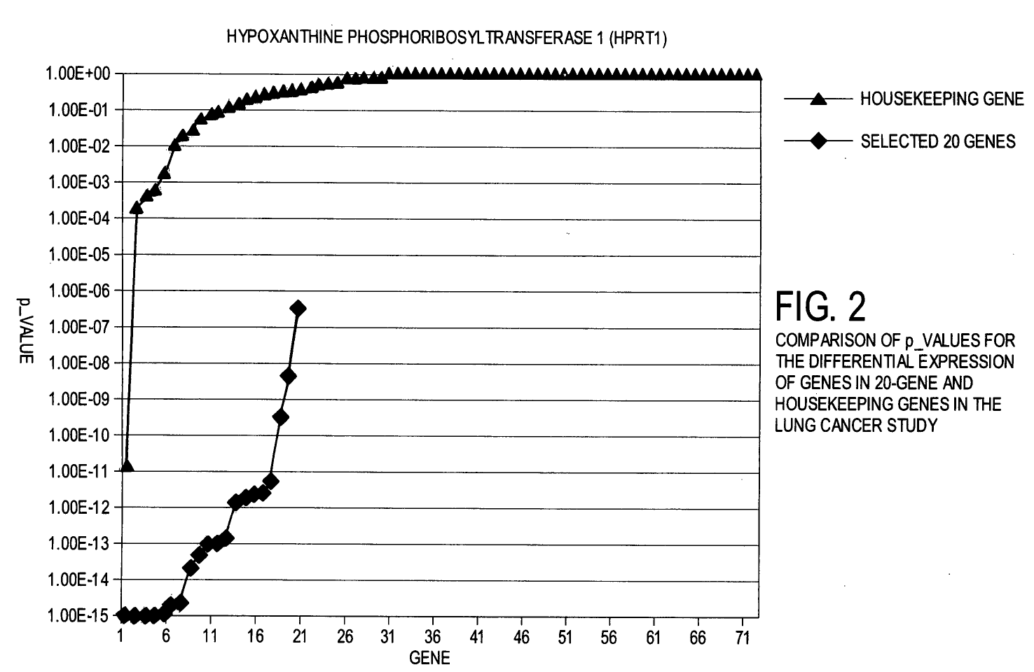

Analysis of variance (ANOVA) was used to determine the fitness of the selected genes and fragments in determining the presence of lung cancer. The method used was similar to that disclosed by Kerr et al. (2000), Analysis of variance for gene expression microarray data, J. Comput. Biol., 7(6):819-837; U.S. Pat. No. 6,344,316 to Lockhart et al.; U.S. Pat. No. 6,322,976 to Aitman et al.; and U.S. Pat. No. 6,258,541 to Chapkin et al., which are incorporated herein by reference. The data were divided into three populations, namely normal lung (n=33), adenocarcinoma (n=25), and squamous cell carcinoma (n=20). ANOVA was used to determine whether the population means differs. The resulting p-value from the ANOVA test is used to determine the confidence level of the selected gene as a marker for NSCLC (the lower the value, the higher the confidence). FIG. 2 shows p-values for the twenty selected genes and fragments compared to those of house keeping genes.

### EXAMPLE 3

### Separation of Normal and Lung Cancer with Expression Profile of the 20 Selected Genes

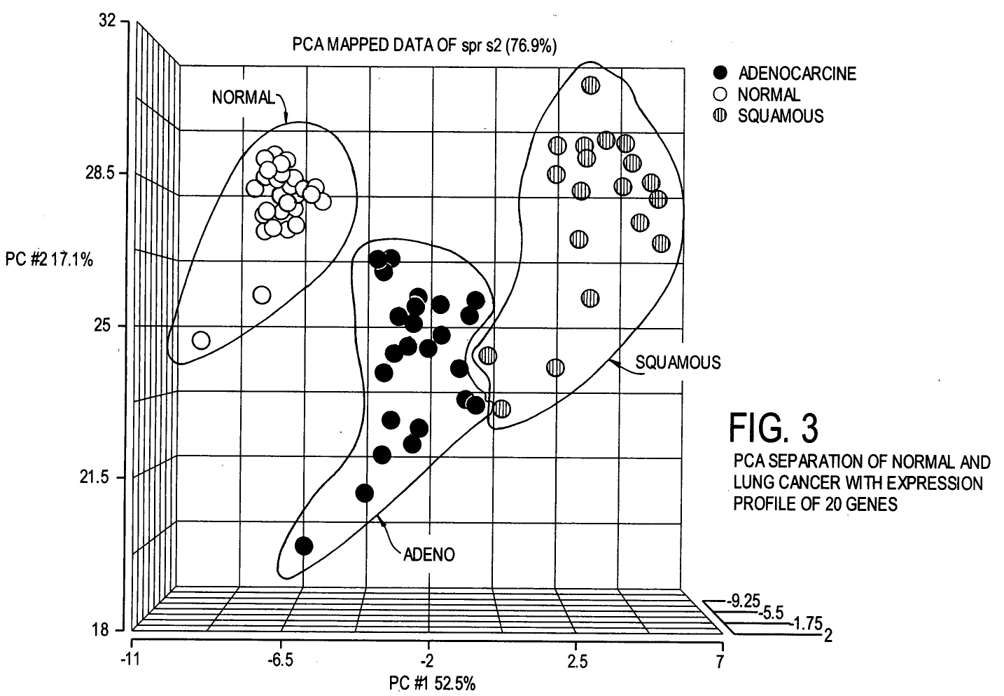

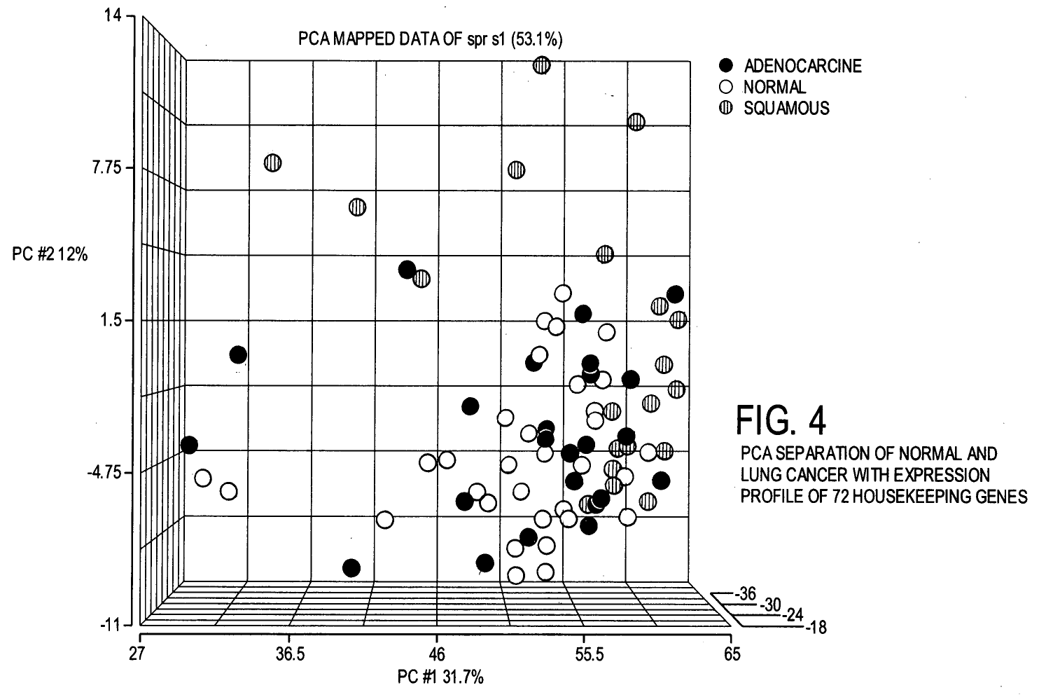

Principle component analysis (PCA) is used to group a set of mixed samples by a set of variables, in this case, the expression levels of the genes and fragments, into normal, adenocarcinoma, and squamous cell carcinoma. PCA is often applied to select a subset of components of the descriptor vectors associated with a set of items that approximates the data within the set. The selected subset of components is typically used to perform analysis of regression and/or correlation on the set of items. Generally, such analysis of regression and correlation both concern the following questions: 1) Does a statistical relation affording some predictability appear between the set of items? 2) How strong is the apparent statistical relation, in the sense of the possible predictive ability that the statistical relation affords? 3) Can a rule be formulated for predicting relations among the set of items, and, if so, how good is this rule? A more detailed description of Principal Component Analysis together with regression analysis and/or correlation analysis may be found in I. T. Jolliffe, Principal Component Analysis, Springer Verlag, New York, 1986 and U.S. Pat. No. 6,349,265 to Pitman et al., which are incorporated herein by reference. FIGS. 3 and 4 shows PCA separation of normal and lung cancer with expression profile of the 20 selected genes (SEQ ID NOS: 1-20) and with 72 house keeping genes, respectively. It is clear from the figures that the 20 selected genes can differentiate between normal lung, adenocarcinoma, squamous cell carcinoma samples while the house keeping gene can not differentiate between normal and tumor samples.

### EXAMPLE 4

### Confounding Factors

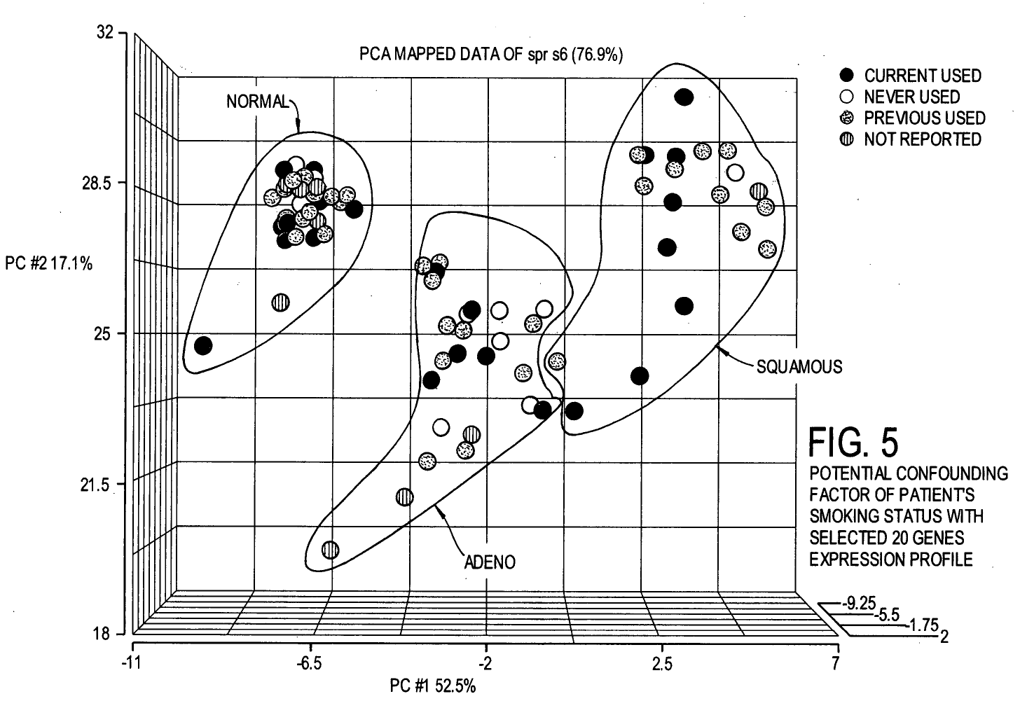

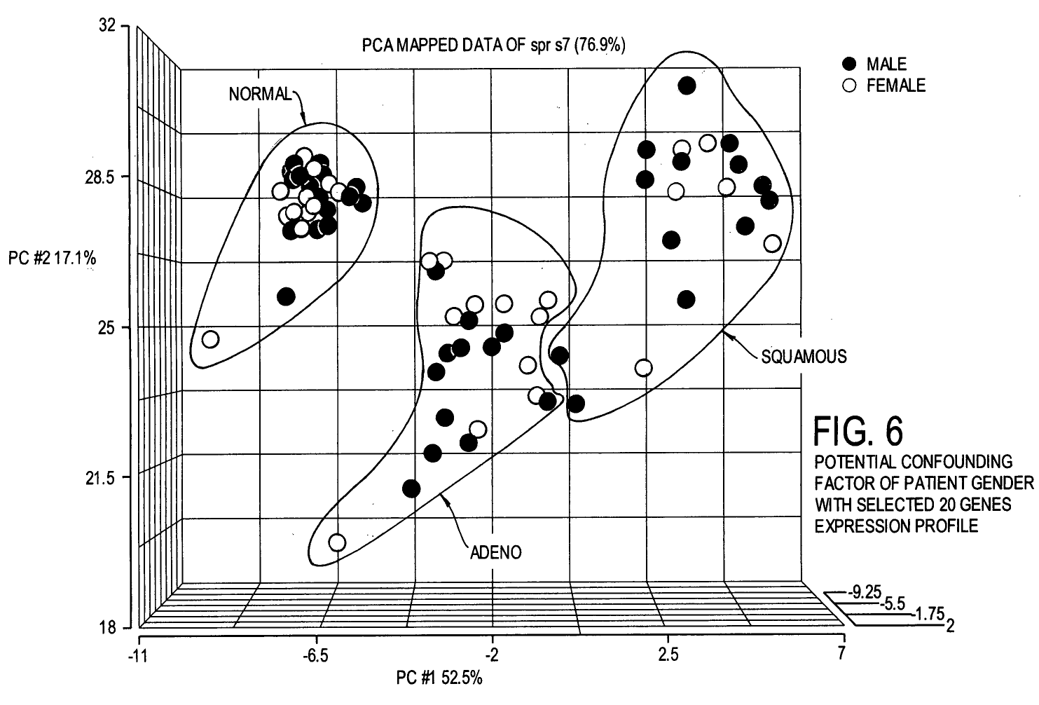

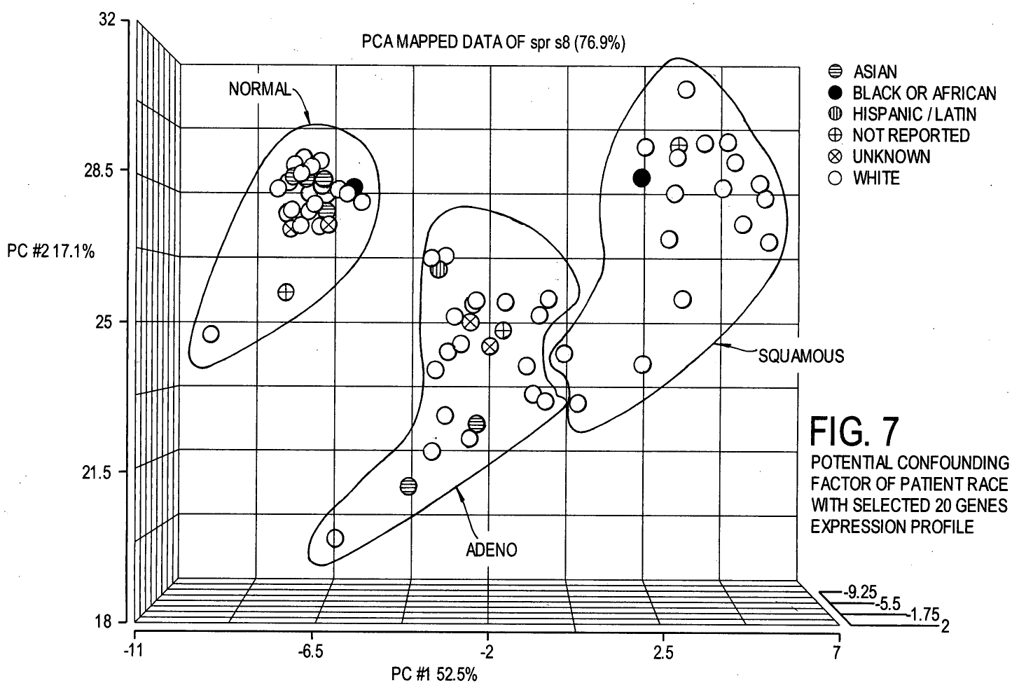

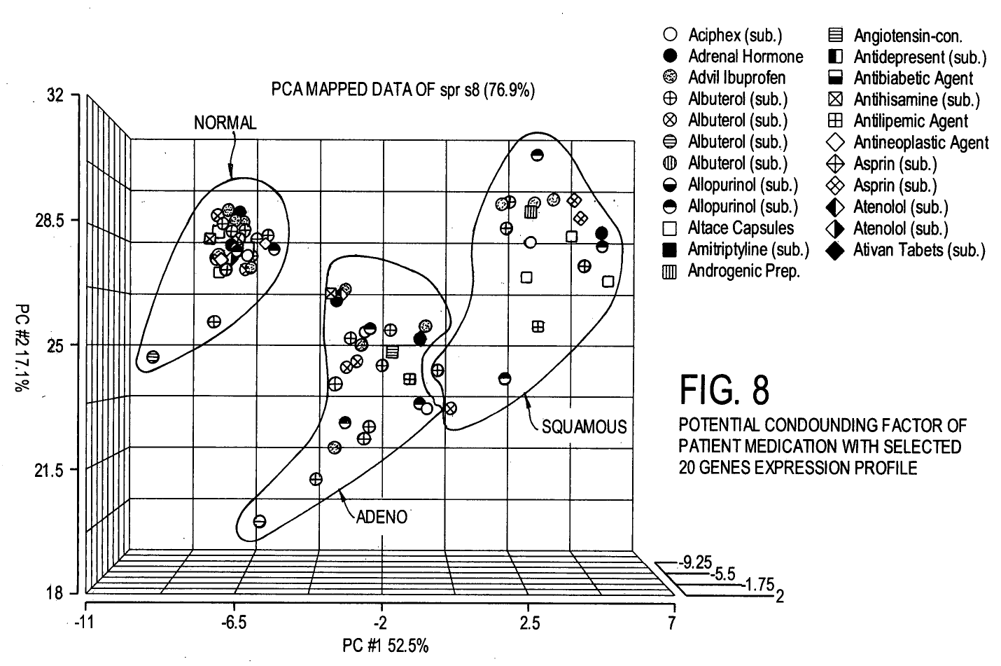

A study of the ability of the 20 selected genes to differentiate between normal and NSCLC samples, when potential confounding factors were present, was examined. The potential confounding factors examined were smoking status (FIG. 5), sex (FIG. 6), race (FIG. 7), and medication status (FIG. 8). FIGS. 5-8 are PCA mapped data for the different confounding factors. It is clear from the results that no confounding factors were present for smoking status, sex, race, and medication status.

### EXAMPLE 5

### Array Design

The MetriGenix 4D Lung Cancer Array monitors the expression activity of 80 genes that are associated with lung cancer. The present invention resides in the identification and/or selection of, from the 80 genes, a smaller, more concise gene group for use in the detection and differentiation of lung cancer. The smaller gene set of the present invention (and associated products) are far more amenable than larger gene groups to a kit format and for the generation and interpretation of recognizable patterns which are the basis of the present invention.

A subset of 20 genes (the 20 selected genes) has been identified whose expression response can be used to distinguish between NSCLC and normal lung tissue. Among the 20 selected genes, 8 genes are over expressed at least two fold in NSCLC and 12 genes are under expressed at least two fold compared to matching normal lung tissues. Some of the genes on the array outside of the 20 gene subset are uniquely modulated in the different types of NSCLC, and can thereby serve as NSCLC-classification markers. The array also included 16 controls, including 3 hybridization controls, 1 negative control, 8 house keeping genes, 3 staining controls and a sample preparation control. All chip probe oligos are printed in duplicates.

### EXAMPLE 6

### Probe Design

The oligonucleotide probes used on the array to hybridize the subset of 20 selected genes are designed using a probe design program that strives to minimize the possibilities that a probe cross hybridizes to genes other than itself and repetitive sequences or sequences with low complexity in the whole gene sequence. Probe design is constrained based on the following selection criteria: length of 58 to 62 nucleotides, melting temperature (Tm) between 70° C. to 80° C., and G/C content is between 35-45%. In vitro transcription (IVT) is a well-adopted method for assay sample preparation that produces antisense sequence; however, IVT has bias to amplify messenger RNA at 3′ end. Accordingly, an additional probe design criteria is to select probes within 500 bases of the 3′ of the gene strand that encodes the open reading frame. All probes are BLAST searched against Genbank or other human gene sequence databases. The probes are sense strands to capture the antisense sequences of the target and are synthesized with an amine linker at the 5′ end for surface immobilization. A preferred set of probes are as designated by SEQ ID NO: 21-40.

### EXAMPLE 7

### Sample Preparation

Total RNA from Normal and Tumor Lung tissue was transformed into cRNA per standard protocols (Lockhart et al., 1996, Nat. Biotech., 14(13):1675-1680). The cRNA is produced with biotinylated CTP and UTP nucleotides, for subsequent streptavidin-horseradish peroxidase staining for indirect detection of hybridization via chemiluminescence. Prior to hybridization each sample is denatured at 95° C. for 5 minutes, vortexed and spun down for two minutes. In a standard array assay, 10 micrograms of cRNA is used per hybridization. Hybridization is carried out in buffer containing 1×MES, 0.88 M NaCl, 0.02 M EDTA, 0.5% Sarcosine, 33% Formamide and 50 μg/ml Herring Sperm DNA.

### EXAMPLE 8

### Array Hybridization and Detection

The array is processed using the MetriGenix Hybridization Station—MGX 2000. The MGX 2000 is an automated microfluidics station that integrates chip conditioning, sample injection, hybridization, blocking and staining. Arrays are conditioned with buffer 1 (1×SSPE, 2.5% Triton X-100) for 5 minutes and then blocked with 1% goat serum in SSPE for 5 minutes. Hybridization is performed at 37° C. for 2 hours. After the hybridization, the sample is removed and the chip is washed with buffer 1 for 5 minutes, followed by another blocking for 5 minutes with 1% goat serum. Staining is performed using 0.75 ng Streptavidin-horseradish peroxidase in 1×SSPE for 5 minutes. Array imaging is performed using the Metrigenix Detection System—MGX 1200CL. The MGX 1200CL uses a CCD camera to detect enzyme catalyzed chemiluminescence under flow of enzymatic substrate. The captured digital image is analyzed to produce relative quantitative values of each genes expression level monitored by the chip.

### EXAMPLE 9

### Differential Expression of the 20 Selected Genes

The differential expression level of genes between samples is determined by calculating the quotient of each individual gene intensity following normalization to a defined control. The control can either be an endogenous constantly expressed gene, e.g. a house keeping gene, or an exogenous gene that has been added to both samples at the same level. Using a known lung tissue normal sample as the denominator term and an endogenous control, GAPDH, a panel of blinded lung tissue samples was assessed using the 20 gene subgroup on the 4D Lung Cancer chip. The panel included 3 NSCLC samples (Tests-1, -2, and -3) and an additional normal (Test-4). As observed in FIG. 9, the normal pattern for the 20 gene subgroup was observed for the normal lung sample (Test-4), and the modulated response was observed for the 3 NSCLC samples (Tests 1, 2, and 3). The normal relative gene expression level for each of the 20 selected genes is defined by the gray bars; the NSCLC relative gene expression level for each of the 20 selected genes is defined the black bars; and the sample responses of Tests-1 to -4 are defined by the individual points. Sample classification is accomplished by determining if the individual gene responses are in better agreement with either the gray or black bars. FIG. 9 shows that Test-4 matches with the normal gene pattern and that Tests 1, 2, and 3 matches with the NSCLC gene pattern indicating a the ability of the 20 genes set to differentiate between NSCLC and normal samples.

### EXAMPLE 10

### Accuracy of Gene Set with Random Gene Removal

Various number of genes (0, 2, 4, 6, 8, 10, 12, or 14) were randomly selected and removed from the 20 gene set. Expression profiles of remained genes for tested 78 lung tissue samples were the used to perform 100 cycles of ⅓ cross-validation. Each number of gene reduction was repeated five times in order to calculate the total average percentage of prediction errors. The result is shown in Table 2.

TABLE 2

| | Number of gene removed | | | | | | | |
|---|---|---|---|---|---|---|---|---|
| | 0 | 2 | 4 | 6 | 8 | 10 | 12 | 14 |
| Average prediction | 0.1 | 0.3 | 1.0 | 1.1 | 1.4 | 3.5 | 6.0 | 10.5 |
| error | | | | | | | | |
| STD | 0 | 0.28 | 0.7 | 0.25 | 0.58 | 1.82 | 2.4 | 2.64 |

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. An isolated gene set having less than about 400 sequences comprising from about 6 to about 20 sequences selected from the group consisting of SEQ ID NOS: 1-20.

2. A kit comprising probes greater than about 30 nucleotides in length that specifically bind to from about 6 to about 20 sequences selected from the group consisting of SEQ ID NOS: 1-20.

3. The kit of claim 2, wherein the probes are selected from the group consisting of SEQ ID NOS: 21-40.

4. A gene chip comprising probes greater than about 30 nucleotides in length that specifically bind to about 6 to about 20 sequences selected from the group consisting of SEQ ID NOS: 1-20.

5. The gene chip of claim 4, wherein the probes are selected from the group consisting of SEQ ID NOS: 21-40.

6. A method for detecting lung cancer comprising
providing a nucleic acid sample from an individual;
hybridizing the nucleic acid sample with probes that specifically hybridize with about 6 to about 20 sequences selected from the group consisting of SEQ ID NOS: 1-20;
detecting a presence of hybridization; and
correlating the presence of hybridization with the presence or absence of lung cancer.

7. The method of claim 6, where in the probes are selected from the group consisting of SEQ ID NOS: 21-40.

8. The method of claim 6, wherein the hybridizing step is performed on a gene chip.

9. A method for differentiating lung cancer types comprising
providing a nucleic acid sample from an individual;
hybridizing the nucleic acid sample with probes that specifically hybridize with about 6 to about 20 sequences selected from the group consisting of SEQ ID NOS: 1-20;
detecting a presence of hybridization; and
correlating the presence of hybridization with the type of lung cancer.

10. The method of claim 9, where in the probes are selected from the group consisting of SEQ ID NOS: 21-40.

11. The method of claim 9, wherein the hybridizing step is performed on a gene chip.

12. A method of monitoring the treatment of a patient with lung cancer comprising
administering a pharmaceutical composition to the patient;
obtaining a nucleic acid sample from the patient;
contacting the tissue sample with probes which specifically hybridize with about 6 to about 20 sequences selected from the group consisting of SEQ ID NOS: 1-20; and
correlating the hybridization pattern with the effectiveness of the pharmaceutical composition in treating lung cancer.

13. The method of claim 12, where in the probes are selected from the group consisting of SEQ ID NOS: 21-40.

14. The method of claim 12, wherein the hybridizing step is performed on a gene chip.

15. A method for screening for an agent capable of modulating the onset or progression of lung cancer comprising
exposing a cell to the agent;
obtaining a nucleic acid sample from the cell;
contacting the nucleic acid sample with probes which specifically hybridize with about 6 to about 20 sequences selected from the group consisting of SEQ ID NOS: 1-20; and
correlating the hybridization pattern with the effectiveness of the agent in modulating the onset or progression of lung cancer.

16. The method of claim 15, where in the probes are selected from the group consisting of SEQ ID NOS: 21-40.

17. The method of claim 15, wherein the hybridizing step is performed on a gene chip.

18. A method for detecting lung cancer comprising
providing a sample from an individual;
contacting the sample with probes that specifically binds gene products of about 6 to about 20 sequences selected from the group consisting of SEQ ID NOS: 1-20;
detecting a binding pattern; and
correlating the binding pattern with the presence or absence of lung cancer.

19. The method of claim 18, where in the probes are selected from the group consisting of SEQ ID NOS: 21-40.

20. The method of claim 18, wherein the contacting step is performed on a gene chip.

21. The method of claim 18, wherein the gene products are selected from the group consisting of DNA, RNA, and proteins.

22. A method for differentiating lung cancer types comprising
providing a sample from an individual;
contacting the sample with probes that specifically binds gene products of about 6 to about 20 sequences selected from the group consisting of SEQ ID NOS: 1-20;
detecting a binding pattern; and
correlating the binding pattern with the type of lung cancer.

23. The method of claim 22, where in the probes are selected from the group consisting of SEQ ID NOS: 21-40.

24. The method of claim 22, wherein the contacting step is performed on a gene chip.

25. The method of claim 22, wherein the gene products are selected from the group consisting of DNA, RNA, and proteins.

26. A method of monitoring the treatment of a patient with lung cancer comprising
administering a pharmaceutical composition to the patient;
obtaining a sample from the patient;
contacting the tissue sample with probes that specifically bind gene products of about 6 to about 20 sequences selected from the group consisting of SEQ ID NOS: 1-20;
detecting a binding pattern; and
correlating the binding pattern with the effectiveness of the pharmaceutical composition in treating lung cancer.

27. The method of claim 26, where in the probes are selected from the group consisting of SEQ ID NOS: 21-40.

28. The method of claim 26, wherein the contacting step is performed on a gene chip.

29. The method of claim 26, wherein the gene products are selected from the group consisting of DNA, RNA, and proteins.

30. A method for screening for an agent capable of modulating the onset or progression of lung cancer comprising
exposing a cell to the agent;
obtaining a sample from the cell;
contacting the sample with probes that specifically bind gene products of about 6 to about 20 sequences selected from the group consisting of SEQ ID NOS: 1-20;
detecting a binding pattern; and
correlating the binding pattern with the effectiveness of the agent in modulating the onset or progression of lung cancer.

31. The method of claim 30, where in the probes are selected from the group consisting of SEQ ID NOS: 21-40.

32. The method of claim 30, wherein the contacting step is performed on a gene chip.

33 The method of claim 30, wherein the gene products are selected from the group consisting of DNA, RNA, and proteins.
